SEQUENCE LISTING (100 sequences)

SEQ ID NO: 1 - protein, 10 aa, Homo sapiens
PGPPDVPDHA

SEQ ID NO: 2 - protein, 13 aa, Homo sapiens
GLPGPPDVPDHAA

SEQ ID NO: 3 - protein, 17 aa, Homo sapiens
TGPIGPGPAGAGDKGES

SEQ ID NO: 4 - protein, 27 aa, Homo sapiens
AGPGAGAPGAPGVGPAGKSGDRGETGP

SEQ ID NO: 5 - protein, 23 aa, Homo sapiens
ANGAGNDGAKGDAGAGAGSQGAG

SEQ ID NO: 6 - protein, 24 aa, Homo sapiens
KGNSGEPGAGSKGDTGAKGEGPVG

SEQ ID NO: 7 - protein, 23 aa, Homo sapiens
ADGQPGAKGEGDAGAKGDAGPGP

SEQ ID NO: 8 - protein, 29 aa, Homo sapiens
GESGREGAGAEGSPGRDGSGAKGDRGETG

SEQ ID NO: 9 - protein, 21 aa, Homo sapiens
ADGQGAKGEGDAGAKGDAGGP

SEQ ID NO: 10 - protein, 23 aa, Homo sapiens
ADGQGAKGEGDAGAKGDAGPGPA

SEQ ID NO: 11 - protein, 16 aa, Homo sapiens
GPGEAGKGEQGVGDLG

SEQ ID NO: 12 - protein, 20 aa, Homo sapiens
NSGEPGAGSKGDTGAKGEGP

SEQ ID NO: 13 - protein, 23 aa, Homo sapiens
DGQPGAKGEPGDAGAKGDAGPPG

SEQ ID NO: 14 - protein, 24 aa, Homo sapiens
ADGQPGAKGEGDAGAKGDAGPGPA

SEQ ID NO: 15 - protein, 31 aa, Homo sapiens
GPGESGREGAGAEGSGRDGSPGAKGDRGETG

SEQ ID NO: 16 - protein, 13 aa, Homo sapiens
SGSGPDGKTGPGP

SEQ ID NO: 17 - protein, 18 aa, Homo sapiens
SGEGAGSKGDTGAKGEGP

SEQ ID NO: 18 - protein, 22 aa, Homo sapiens
ADGQGAKGEGDAGAKGDAGPGP

SEQ ID NO: 19 - protein, 16 aa, Homo sapiens
DGEAGKPGRGERGPPG

SEQ ID NO: 20 - protein, 18 aa, Homo sapiens
NSGEGAGSKGDTGAKGEG

SEQ ID NO: 21 - protein, 20 aa, Homo sapiens
GNSGEGAGSKGDTGAKGEGP

SEQ ID NO: 22 - protein, 19 aa, Homo sapiens
NSGEGAGSKGDTGAKGEGP

SEQ ID NO: 23 - protein, 15 aa, Homo sapiens
DGEAGKGRGERGPPG

SEQ ID NO: 24 - protein, 17 aa, Homo sapiens
DGAKGDAGAGAGSQGAG

SEQ ID NO: 25 - protein, 19 aa, Homo sapiens
NGDDGEAGKPGRPGERGGP

SEQ ID NO: 26 - protein, 20 aa, Homo sapiens
AEGSGRDGSGAKGDRGETGP

SEQ ID NO: 27 - protein, 21 aa, Homo sapiens
KGNSGEGAGSKGDTGAKGEGP

SEQ ID NO: 28 - protein, 14 aa, Homo sapiens
SGSPGPDGKTGPGP

SEQ ID NO: 29 - protein, 36 aa, Homo sapiens
GPGADGQPGAKGEGDAGAKGDAGPGPAGPAGPGPIG

SEQ ID NO: 30 - protein, 17 aa, Homo sapiens
EGSGRDGSGAKGDRGET

SEQ ID NO: 31 - protein, 21 aa, Homo sapiens
DGQGAKGEGDAGAKGDAGPPG

SEQ ID NO: 32 - protein, 9 aa, Homo sapiens
GDRGEGPGP

SEQ ID NO: 33 - protein, 15 aa, Homo sapiens
SGRDGSGAKGDRGET

SEQ ID NO: 34 - protein, 17 aa, Homo sapiens
GPGEAGKGEQGVPGDLG

SEQ ID NO: 35 - protein, 19 aa, Homo sapiens
GNSGEGAGSKGDTGAKGEG

SEQ ID NO: 36 - protein, 18 aa, Homo sapiens
NDGAKGDAGAGAGSQGAG

SEQ ID NO: 37 - protein, 19 aa, Homo sapiens
EGSGRDGSGAKGDRGETGP

SEQ ID NO: 38 - protein, 10 aa, Homo sapiens
AGDRGEGPGP

SEQ ID NO: 39 - protein, 17 aa, Homo sapiens
NSGEGAGSKGDTGAKGE

SEQ ID NO: 40 - protein, 18 aa, Homo sapiens
GDDGEAGKPGRGERGPGP

SEQ ID NO: 41 - protein, 20 aa, Homo sapiens
NSGEGAGSKGDTGAKGEGVG

SEQ ID NO: 42 - protein, 26 aa, Homo sapiens
RGPGPGKNGDDGEAGKPGRPGERGGP

SEQ ID NO: 43 - protein, 26 aa, Homo sapiens
ERGSGPAGPKGSGEAGRGEAGLGAKG

SEQ ID NO: 44 - protein, 26 aa, Homo sapiens
KGDRGETGPAGPGAGAPGAGPVGPAG

SEQ ID NO: 45 - protein, 17 aa, Homo sapiens
GDDGEAGKGRPGERGPG

SEQ ID NO: 46 - protein, 15 aa, Homo sapiens
PGEAGKGEQGVGDLG

SEQ ID NO: 47 - protein, 13 aa, Homo sapiens
NDGAKGDAGAGAG

SEQ ID NO: 48 - protein, 11 aa, Homo sapiens
SGPDGKTGPGP

SEQ ID NO: 49 - protein, 19 aa, Homo sapiens
GEGAGSKGDTGAKGEGPVG

SEQ ID NO: 50 - protein, 18 aa, Homo sapiens
PGEAGKGEQGVGDLGAGP

SEQ ID NO: 51 - protein, 18 aa, Homo sapiens
GFQGPGEGEPGASGPMGR

SEQ ID NO: 52 - protein, 13 aa, Homo sapiens
KGEQGVGDLGAGP

SEQ ID NO: 53 - protein, 36 aa, Homo sapiens
GPGAGSAGARGAGERGETGPPGPAGFAGPPGADGQP

SEQ ID NO: 54 - protein, 12 aa, Homo sapiens
NGNPGPGPGPSG

SEQ ID NO: 55 - protein, 15 aa, Homo sapiens
DGAGKNGERGGGGGP

SEQ ID NO: 56 - protein, 21 aa, Homo sapiens
GGGSDGKPGGSQGESGRPGPG

SEQ ID NO: 57 - protein, 20 aa, Homo sapiens
DAGAGAGGKGDAGAGERGPG

SEQ ID NO: 58 - protein, 10 aa, Homo sapiens
AGAGHPGPGP

SEQ ID NO: 59 - protein, 22 aa, Homo sapiens
KGDAGAGAGGKGDAGAGERGPG

SEQ ID NO: 60 - protein, 34 aa, Homo sapiens
GKDGGPAGNTGAPGSGVSGPKGDAGQPGEKGSPG

SEQ ID NO: 61 - protein, 9 aa, Homo sapiens
SGERGETGP

SEQ ID NO: 62 - protein, 26 aa, Homo sapiens
ERGEAGIGVGAKGEDGKDGSGEGANG

SEQ ID NO: 63 - protein, 22 aa, Homo sapiens
GQNGEGGKGERGAGEKGEGGPG

SEQ ID NO: 64 - protein, 12 aa, Homo sapiens
GFGPQGDRGFPG

SEQ ID NO: 65 - protein, 35 aa, Homo sapiens
GLGDPGASYGRNGRDGERGPGVAGIPGVPGPPGPG

SEQ ID NO: 66 - protein, 25 aa, Homo sapiens
GMPGADGPPGHPGKEGGEKGGQGPG

SEQ ID NO: 67 - protein, 9 aa, Homo sapiens
GPGPGPGPA

SEQ ID NO: 68 - protein, 56 aa, Homo sapiens
GDPGPVGEPGAMGLPGLEGFPGVKGDRGPAGPGIAGMSGKPGAGPGVGEPGERGPV

SEQ ID NO: 69 - protein, 28 aa, Homo sapiens
PGGEPGTDGAAGKEGPGKQGFYGPGPKG

SEQ ID NO: 70 - protein, 29 aa, Homo sapiens
AAGEPGPHGGVPGSVGPKGSSGSPGQGPG

SEQ ID NO: 71 - protein, 20 aa, Homo sapiens
GIPSGSEGGSSSTMYVSGGP

SEQ ID NO: 72 - protein, 12 aa, Homo sapiens
SVPGPGPGPGPG

SEQ ID NO: 73 - protein, 10 aa, Homo sapiens
GMPGPGPGPG

SEQ ID NO: 74 - protein, 19 aa, Homo sapiens
GSQGFGYGEQGPGPGPEGP

SEQ ID NO: 75 - protein, 24 aa, Homo sapiens
GKSGPSGQTGDPGLQGSGPGEGFG

SEQ ID NO: 76 - protein, 31 aa, Homo sapiens
PGVPGEPGKGEQGLMGPLGPPGQKGSIGAPG

SEQ ID NO: 77 - protein, 27 aa, Homo sapiens
VPGPKGSGHPGMPGGMGTPGEPGPQGP

SEQ ID NO: 78 - protein, 9 aa, Homo sapiens
GPGPGPGPS

SEQ ID NO: 79 - protein, 24 aa, Homo sapiens
GPGPGYGSQGIKGEQGQGFPGKGT

SEQ ID NO: 80 - protein, 30 aa, Homo sapiens
RGPPGPGRDGEDGPTGPPGPGPPGPGLGGN

SEQ ID NO: 81 - protein, 31 aa, Homo sapiens
RTGEVGAVGPGFAGEKGPSGEAGTAGPGTGP

SEQ ID NO: 82 - protein, 16 aa, Homo sapiens
GGPPGRDGEDGTGPGP

SEQ ID NO: 83 - protein, 21 aa, Homo sapiens
GAGPGKAGEDGHGKPGRGERG

SEQ ID NO: 84 - protein, 17 aa, Homo sapiens
GEKGSGEAGTAGPGTGP

SEQ ID NO: 85 - protein, 32 aa, Homo sapiens
TGEVGAVGPGFAGEKGPSGEAGTAGPGTGPQG

SEQ ID NO: 86 - protein, 12 aa, Homo sapiens
GPGPGRDGEDGP

SEQ ID NO: 87 - protein, 28 aa, Homo sapiens
QGPGPPGSGPALEGPKGNPGPQGPGRPG

SEQ ID NO: 88 - protein, 19 aa, Homo sapiens
GSGSRGAPGQYGEKGFGDP

SEQ ID NO: 89 - protein, 15 aa, Homo sapiens
SQLQKVPPEWKALTD

SEQ ID NO: 90 - protein, 11 aa, Homo sapiens
ELERPGGNEIT

SEQ ID NO: 91 - protein, 13 aa, Homo sapiens
QLGGPEAAKSDET

SEQ ID NO: 92 - protein, 25 aa, Homo sapiens
GGLGETEDLLSFEAYELRTDSWTHL

SEQ ID NO: 93 - protein, 12 aa, Homo sapiens
HSHEDMLVVDPK

SEQ ID NO: 94 - protein, 16 aa, Homo sapiens
DHDVGSELPPEGVLGA

SEQ ID NO: 95 - protein, 16 aa, Homo sapiens
VSQVGRASLQHGQAAE

SEQ ID NO: 96 - protein, 26 aa, Homo sapiens
KKTETQEKNPLPSKETIEQEKQAGES

SEQ ID NO: 97 - protein, 25 aa, Homo sapiens
KTETQEKNPLPSKETIEQEKQAGES

SEQ ID NO: 98 - protein, 24 aa, Homo sapiens
TETQEKNPLPSKETIEQEKQAGES

SEQ ID NO: 99 - protein, 44 aa, Homo sapiens
MSDKPDMAEIEKFDKSKLKKTETQEKNPLPSKETIEQEKQAGES

SEQ ID NO: 100 - protein, 4 aa, Homo sapiens
SDKP

(19) United States
(12) Patent Application Publication
(10) Pub. No.: US 2022/0050113 A1
(43) Pub. Date: Feb. 17, 2022

(54) USE OF AMNIOTIC FLUID PEPTIDES FOR PREDICTING POSTNATAL RENAL FUNCTION IN CONGENITAL ANOMALIES OF THE KIDNEY AND THE URINARY TRACT

(71) Applicants: INSERM (Institut National de la Santé et de la Recherche Médicale), Paris (FR); Université Paul Sabatier Toulouse III, Toulouse (FR); Centre Hospitalier Universitaire de Toulouse, Toulouse (FR)

(72) Inventors: Joost SCHANSTRA, Toulouse Cedex 4 (FR); Julie KLEIN, Toulouse Cedex 4 (FR); Benjamin BREUIL, Toulouse Cedex 4 (FR); Stéphane DECRAMER, Toulouse (FR); Bénédicte BUFFIN-MEYER, Toulouse Cedex 4 (FR)

(21) Appl. No.: 17/275,364

(22) PCT Filed: Sep. 13, 2019
(86) PCT No.: PCT/EP2019/074472

§ 371 Date: Mar. 11, 2021

(30) Foreign Application Priority Data
Sep. 14, 2018 (EP) 18306197.7

(51) Int. Cl. G01N 33/68; G16B 40/20

(52) U.S. Cl. CPC G01N 33/689; G01N 2800/385; G01N 2800/347; G16B 40/20

(57) ABSTRACT

Bilateral congenital anomalies of the kidney and urinary tract (CAKUT) are the main cause of childhood chronic kidney disease (CKD). Accurate and non-biased prenatal prediction of postnatal disease evolution is currently lacking, but is essential for prenatal counseling and disease management. Here the inventors aimed to develop an objective and quantifiable risk prediction method based on amniotic fluid (AF) peptides. 178 fetuses with bilateral CAKUT were included in a prospective multicenter study. The AF peptide content was studied using capillary electrophoresis coupled to mass spectrometry. The endpoint was early-onset renal failure (CKD stage 3-5) or death due to end-stage renal disease at two years of age. Among the ˜7000 peptide candidates, 98 were associated with early severe renal failure. The most frequently found peptides associated with severe disease were fragments from extracellular matrix proteins and thymosin-P4. Combination of those 98 peptides in a classifier lead to the prediction of postnatal renal outcome in a blinded validation set of 51 patients with a 88% (95% CI: 64-98) sensitivity, 97% (95% CI: 85-100) specificity and an AUC of 0.96 (95% CI: 0.87-1.00), outperforming predictions based on currently used clinical methods. The classifier also predicted normal postnatal renal function in 75% of terminated pregnancies where fetopathology showed kidneys compatible with normal life. Analysis of AF peptides thus allows a precise and quantifiable prediction of postnatal renal function in bilateral CAKUT with potential major impact on pre- and postnatal disease management.

### FIELD OF THE INVENTION

The present invention relates to the use of amniotic fluid peptides for predicting postnatal renal function in congenital anomalies of the kidney and the urinary tract.

### BACKGROUND OF THE INVENTION

Obstetricians are frequently confronted with congenital anomalies of the kidney and the urinary tract (CAKUT), which represent 20-30% of all inborn malformations¹. Whereas prognosis is generally good in unilateral disease, bilateral CAKUT is the predominant cause of chronic kidney disease (CKD) in childhood² and accounts for ˜50% of pediatric and young adult end stage renal disease (ESRD) cases³.

Bilateral CAKUT displays a wide spectrum of outcomes ranging from death in utero to normal renal function after birth. Unfortunately postnatal renal outcome is difficult to predict in many cases. In monogenic CAKUT cases a clear genotype-phenotype correlation is absent^{1,4}. Likewise, postnatal renal function cannot be predicted from the prenatal sonographic appearance, except in extreme cases (e.g. bilateral agenesis)^{5,6}. Finally, invasive testing such as assessing fetal serum β2-microglobulin⁷ is rather controversial due to the absence of clear cutoff values and the fact that only measurements at advanced gestational age are predictive^{8,9}. Hence, the currently available parameters have low to moderate predictive value at best in the assessment of the risk of CAKUT fetuses to develop severe CKD.

This predictive uncertainty has particularly serious implications for prenatal counseling of the parents confronted with the issue of elective termination of pregnancy. Such uncertainty leads to situations where half of the cases of severe bilateral CAKUT for whom termination of pregnancy was considered but not performed had normal kidney function at a median age of 29 months¹⁰. In addition, knowledge of the precise outcome would allow anticipating dialysis, transplantation or palliative care in ongoing pregnancies. Therefore methods using quantifiable and more objective parameters are necessary to faithfully predict, in utero, postnatal renal function in bilateral CAKUT.

The absence of a clear genotype-phenotype correlation in CAKUT^{1,4} suggests that searching markers of progression should focus on traits beyond the genotype, closer to the phenotype. In small proof-of-concept studies, we have shown that peptides in fetal body fluid (urine or amniotic fluid (AF)) allow prediction of renal and neurological postnatal outcome in fetuses with posterior urethral valves (PUV)¹¹ and in fetuses infected with cytomegalovirus¹² respectively, outperforming ultrasound and biochemical parameters. This laid the groundwork for the potential use of fetal body fluid peptides in predicting disease progression in prenatal medicine.

### SUMMARY OF THE INVENTION

The present invention relates to the use of amniotic fluid peptides for predicting postnatal renal function in congenital anomalies of the kidney and the urinary tract. In particular, the present invention is defined by the claims.

### DETAILED DESCRIPTION OF THE INVENTION

Bilateral congenital anomalies of the kidney and urinary tract (CAKUT) are the main cause of childhood chronic kidney disease (CKD). Accurate and non-biased prenatal prediction of postnatal disease evolution is currently lacking, but is essential for prenatal counseling and disease management. Here the inventors aimed to develop an objective and quantifiable risk prediction method based on amniotic fluid (AF) peptides. 178 fetuses with bilateral CAKUT were included in a prospective multicenter study. The AF peptide content was studied using capillary electrophoresis coupled to mass spectrometry. The endpoint was early-onset renal failure (CKD stage 3-5) or death due to end-stage renal disease at two years of age. Among the ˜7000 peptide candidates, 98 were associated with early severe renal failure. The most frequently found peptides associated with severe disease were fragments from extracellular matrix proteins and thymosin-β4. Combination of those 98 peptides in a classifier lead to the prediction of postnatal renal outcome in a blinded validation set of 51 patients with a 88% (95% CI: 64-98) sensitivity, 97% (95% CI: 85-100) specificity and an AUC of 0.96 (95% CI: 0.87-1.00), outperforming predictions based on currently used clinical methods. The classifier also predicted normal postnatal renal function in 75% of terminated pregnancies where fetopathology showed kidneys compatible with normal life. Analysis of AF peptides thus allows a precise and quantifiable prediction of postnatal renal function in bilateral CAKUT with potential major impact on pre- and postnatal disease management (ClinicalTrials.gov number, NCT02675686).

Methods Involving at Least One Peptide:

Accordingly, the first object of the present invention relates to a method for predicting postnatal renal function in a fetus diagnosed with bilateral congenital anomalies of the kidney and the urinary tract comprising quantifying in a an amniotic fluid sample obtained from the mother the level of at least one peptide of Table A.

By the expression “is at risk of postnatal renal dysfunction” it is meant that the fetus has a high probability of developing chronic kidney disease after birth. In particular, it is meant that the fetus has a probability of at least 85% (i.e. 85, 86, 87, 88, 89, 90, 91, 92, 93, 94, 95, 96, 97, 98, 99 or 100%) of developing postnatal dysfunction. The clinical admitted definition of CKD includes all individuals with markers of kidney damage such as albuminuria (ACR, >3 mg/mmol), proteinuria (>15 mg/mmol), haematuria, electrolyte abnormalities due to tubular disorders, renal histological abnormalities, structural abnormalities detected by imaging or a history of kidney transplantation or those with a glomerular filtration rate (GFR) of less than 60 ml/min/1.73 m² on at least 2 occasions 90 days apart (with or without markers of kidney damage).

According to the present invention, the peptides of the invention are characterized by the amino acid sequences reported in Table A.

In some embodiments, the levels of at least 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97 or 98 peptides from Table A are determined in the amniotic fluid sample.

In some embodiments, the level of peptide 31862 is determined in the amniotic fluid sample (Table 2).

In some embodiments, the levels of 2 peptides selected in the group consisting of peptides 4697, 5420, 6196, 6400, 6600, 7437, 8721, 15510, 17010, 17207, 17264, 19221, 20228, 21320, 21342, 21353, 21684, 21830, 22456, 23894, 24856, 24868, 26070, 27115, 29894, 31787, 32876, 33930, 34055, 35853, 36447, 36627, 41269, 42122, and 45055 are determined in the amniotic fluid sample. In some embodiment the levels of 2 peptides as depicted in Table 3 are determined in the amniotic fluid sample.

In some embodiments, the levels of 3 peptides selected in the group consisting of peptides 2029, 4727, 5019, 5116, 5781, 7823, 10250, 10640, 11078, 14475, 15732, 16805, 17301, 17453, 18627, 18649, 18837, 20863, 20876, 21028, 21956, 22377, 22992, 23789, 24148, 24608, 25060, 25800, 29880, 31488, 32038, 33880, 34805, 35226, 35677, 36283, 37285, 37566, 40022, and 64283 are determined in the amniotic fluid sample. In some embodiment the levels of 3 peptides as depicted in Table 4 are determined in the amniotic fluid sample.

In some embodiments, the method of the present invention further comprises measuring at least one clinical parameter. Typically said clinical parameter is selected from the group consisting of Age, gestational age at AF sampling; AF, amniotic fluid volume; bCAKUTPep-Age, combination of the bCAKUTPep classifier with gestational age at sampling; bCAKUTPep-AF, combination of the bCAKUTPep classifier with AF volume;

bCAKUTPep-AF/Age, combination of the bCAKUTPep classifier with both gestational age at sampling and AF volume. In some embodiments, the method of the present invention further comprises determining the amniotic fluid volume (AF).

In some embodiments, the level of 1 peptide selected in the group consisting of peptides 4727, 6400, 6600, 10786, 17760, 21342, 21684, 31862, and 45055 is combined with amniotic fluid volume (AF) for predicting postnatal renal function. In some embodiment the levels of 1 peptide as depicted in Table 5 in combination with amniotic fluid volume (AF) are measured for predicting postnatal renal function.

In some embodiments, the levels of 2 peptides selected in the group consisting of peptides 2029, 3917, 4697, 4793, 5019, 5116, 5420, 5781, 6196, 7437, 7823, 8721, 10250, 10640, 11078, 13891, 14475, 14735, 15510, 15732, 15884, 16197, 16805, 17010, 17207, 17264, 17301, 17453, 18627, 18649, 18837, 19221, 19732, 19950, 20228, 20643, 20863, 20876, 21028, 21076, 21320, 21353, 21830, 21938, 21956, 22377, 22456, 22992, 23577, 23789, 23894, 24148, 24421, 24608, 24856, 24868, 25060, 25170, 25301, 25800, 26070, 27115, 28628, 29880, 29894, 31488, 31787, 32038, 32876, 33930, 34055, 34805, 35226, 35677, 35853, 36283, 36447, 36627, 37285, 37566, 37690, 40022, 41269, 42122, 42214, 64283 are combined with amniotic fluid volume (AF) for predicting postnatal renal function. In some embodiment the levels of 2 peptides as depicted in Table 6 in combination with amniotic fluid volume (AF) are measured for predicting postnatal renal function.

Methods Involving the Measurement of Thymosin-β4 or Fragment Thereof:

A further object of the present invention relates to a method for predicting postnatal renal function in a fetus diagnosed with bilateral congenital anomalies of the kidney and the urinary tract comprising quantifying in a an amniotic fluid sample obtained from the mother the level of thymosin-b4 or a fragment thereof.

As used herein, the term “thymosin-β4” has its general meaning in the art and refers to the polypeptide having the amino acid sequence as set forth in SEQ ID NO:99.

>sp|P62328|TYB4_HUMAN Thymosin beta-4
OS = Homo sapiens
OX = 9606 GN = TMSB4X PE = 1 SV = 2
SEQ ID NO: 99
MSDKPDMAEIEKFDKSKLKKTETQEKNPLPSKETIEQ
EKQAGES

In some embodiments, the level of Ac-SDKP is determined in the amniotic fluid sample.

As used herein, the term “Ac-SDKP” has its general meaning in the art and refers to the polypeptide having the amino acid sequence as set forth in SEQ ID NO:100 (N-acetyl-Ser-Asp-Lys-Pro).

In some embodiments, the fragments are selected from the group consisting of peptides 35677, 33930 and 31862 as depicted in Table A.

In some embodiments, the method of the present invention further comprises measuring at least one clinical parameter. Typically said clinical parameter is selected from the group consisting of Age, gestational age at AF sampling; AF, amniotic fluid volume; bCAKUTPep-Age, combination of the bCAKUTPep classifier with gestational age at sampling; bCAKUTPep-AF, combination of the bCAKUTPep classifier with AF volume; bCAKUTPep-AF/Age, combination of the bCAKUTPep classifier with both gestational age at sampling and AF volume. In some embodiments, the method of the present invention further comprises determining the amniotic fluid volume (AF).

Methods for Determining the Level of the Peptides or Proteins of the Present Invention:

According to the present invention, the level of the peptide, protein, or protein fragment in the amniotic fluid sample is determined by any conventional method or assay well known in the art.

Standard methods of determining the level of a soluble marker typically involve contacting the sample obtained from the patient with a binding partner specific for said marker. In some embodiments, the binding partner may be an antibody that may be polyclonal or monoclonal, preferably monoclonal, directed against the specific soluble marker. Polyclonal antibodies of the invention or a fragment thereof can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies of the invention or a fragment thereof can be prepared and isolated using any technique that provides for the production of antibody molecules by continuous cell lines in culture. Techniques for production and isolation include but are not limited to the hybridoma technique; the human B-cell hybridoma technique; and the EBV-hybridoma technique. In some embodiments, the binding partner may be an aptamer. Aptamers are a class of molecule that represent an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library. In some embodiments, the binding partner of the invention is labelled with a detectable molecule or substance, such as a chromogenic substrate, a fluorescent molecule, a radioactive molecule or any other labels known in the art. Labels are known in the art that generally provide (either directly or indirectly) a signal. As used herein, the term “labelled”, with regard to the antibody or aptamer, is intended to encompass direct labelling of the antibody or aptamer by coupling (i.e., physically linking) a detectable substance, such as a radioactive agent, an enzyme (e.g. horseradish peroxidase, or alkaline phosphatase) or a fluorophore (e.g. fluorescein isothiocyanate (FITC) or phycoerythrin (PE) or Indocyanine (Cy5) or allophycocyanin) to the antibody or aptamer, as well as indirect labelling of the probe or antibody by reactivity with a detectable substance. An antibody or aptamer of the invention may be labelled with a radioactive molecule by any method known in the art. For example radioactive molecules include but are not limited to radioactive atom for scintigraphic studies such as I¹²³, I¹²⁴, In¹¹¹, Re¹⁸⁶, Re¹⁸⁸. Preferably, the antibodies against the surface markers are already conjugated to a fluorophore (e.g. FITC-conjugated and/or PE-conjugated or allophycocyanin). Methods for labeling biological molecules such as antibodies are well-known in the art (see, for example, “Affinity Techniques. Enzyme Purification: Part B”, Methods in EnzymoL, 1974, Vol. 34, W. B. Jakoby and M. Wilneck (Eds.), Academic Press: New York, N.Y.; and M. Wilchek and E. A. Bayer, Anal. Biochem., 1988, 171: 1-32). The aforementioned assays may involve the binding of the binding partners (i.e. antibodies or aptamers) to a solid support. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like. The solid surfaces are preferably beads. Since extracellular vesicles have a diameter of roughly 0.1 to 1 μm, the beads for use in the present invention should have a diameter larger than 1 μm. Beads may be made of different materials, including but not limited to glass, plastic, polystyrene, and acrylic. In addition, the beads are preferably fluorescently labelled.

Examples of assays include competition assays, direct reaction assays sandwich-type assays and immunoassays (e.g. ELISA). The assays may be quantitative or qualitative. There are a number of different conventional assays for detecting formation of an antibody-peptide complex. For example, the detecting step can comprise performing an ELISA assay, performing a lateral flow immunoassay, performing an agglutination assay, analyzing the sample in an analytical rotor, or analyzing the sample with an electrochemical, optical, or opto-electronic sensor. These different assays are well-known to those skilled in the art. For example, any of a number of variations of the sandwich assay technique may be used to perform an immunoassay. Briefly, in a typical sandwich assay, a first antibody specific for the peptide or protein is immobilized on a solid surface and the sample to be tested is brought into contact with the immobilized antibody for a time and under conditions allowing formation of the immunocomplex. Following incubation, a second antibody of the present invention that is labeled with a detectable moiety is added and incubated under conditions allowing the formation of a ternary complex between any immunocomplex and the labeled antibody. Any unbound material is washed away, and the presence of peptide or protein in the sample is determined by observation/detection of the signal directly or indirectly produced by the detectable moiety. The most commonly used detectable moieties in immunoassays are enzymes and fluorophores. In the case of an enzyme immunoassay (EIA or ELISA), an enzyme such as horseradish perodixase, glucose oxidase, beta-galactosidase, alkaline phosphatase, and the like, is conjugated to the second antibody, generally by means of glutaraldehyde or periodate. The substrates to be used with the specific enzymes are generally chosen for the production of a detectable color change, upon hydrolysis of the corresponding enzyme. In the case of immunofluorescence, the second antibody is chemically coupled to a fluorescent moiety without alteration of its binding capacity. After binding of the fluorescently labeled antibody to the immunocomplex and removal of any unbound material, the fluorescent signal generated by the fluorescent moiety is detected, and optionally quantified. Alternatively, the second antibody may be labeled with a radioisotope, a chemiluminescent moiety, or a bio luminescent moiety. In some embodiments, the assay utilizes a solid phase or substrate to which the antibody of the present invention is directly or indirectly attached. The attachment can be covalent or non-covalent, and can be facilitated by a moiety associated with the polypeptide that enables covalent or non-covalent binding, such as a moiety that has a high affinity to a component attached to the carrier, support or surface. In some embodiments, the substrate is a bead, such as a colloidal particle (e.g., a colloidal nanoparticle made from gold, silver, platinum, copper, metal composites, other soft metals, core-shell structure particles, or hollow gold nanospheres) or other type of particle (e.g., a magnetic bead or a particle or nanoparticle comprising silica, latex, polystyrene, polycarbonate, polyacrylate, or PVDF). Such particles can comprise a label (e.g., a colorimetric, chemiluminescent, or fluorescent label) and can be useful for visualizing the location of the polypeptides during immunoassays. In some embodiments, the substrate is a dot blot or a flow path in a lateral flow immunoassay device. For example, the antibody of the present invention can be attached or immobilized on a porous membrane, such as a PVDF membrane (e.g., an Immobilon™ membrane), a nitrocellulose membrane, polyethylene membrane, nylon membrane, or a similar type of membrane. In some embodiments, the substrate is a flow path in an analytical rotor. In some embodiments, the substrate is a tube or a well, such as a well in a plate (e.g., a microtiter plate) suitable for use in an ELISA assay. Such substrates can comprise glass, cellulose-based materials, thermoplastic polymers, such as polyethylene, polypropylene, or polyester, sintered structures composed of particulate materials (e.g., glass or various thermoplastic polymers), or cast membrane film composed of nitrocellulose, nylon, polysulfone, or the like. A substrate can be sintered, fine particles of polyethylene, commonly known as porous polyethylene, for example, 0.2-15 micron porous polyethylene from Chromex Corporation (Albuquerque, N. Mex.). All of these substrate materials can be used in suitable shapes, such as films, sheets, or plates, or they may be coated onto or bonded or laminated to appropriate inert carriers, such as paper, glass, plastic films, or fabrics. Suitable methods for immobilizing peptides on solid phases include ionic, hydrophobic, covalent interactions and the like.

In some embodiments, the level of the peptide is determined by mass spectrometry. As used herein, the term “mass spectrometry” or “MS” refers to an analytical technique to identify compounds by their mass. MS refers to methods of filtering, detecting, and measuring ions based on their m/z. MS technology generally includes (1) ionizing the compounds to form charged species (e.g., ions); and (2) detecting the molecular weight of the ions and calculating their m/z. The compounds may be ionized and detected by any suitable means. A “mass spectrometer” generally includes an ionizer and an ion detector. In general, one or more molecules of interest are ionized, and the ions are subsequently introduced into a mass spectrographic instrument where, due to a combination of magnetic and electric fields, the ions follow a path in space that is dependent upon mass (“m”) and charge (“z”). See, e.g., U.S. Pat. No. 6,204,500, entitled “Mass Spectrometry From Surfaces;” U.S. Pat. No. 6,107,623, entitled “Methods and Apparatus for Tandem Mass Spectrometry;” U.S. Pat. No. 6,268,144, entitled “DNA Diagnostics Based On Mass Spectrometry;” U.S. Pat. No. 6,124,137, entitled “Surface-Enhanced Photolabile Attachment And Release For Desorption And Detection Of Analytes;” Wright et al., Prostate Cancer and Prostatic Diseases 2:264-76 (1999); and Merchant and Weinberger, Electrophoresis 21:1164-67 (2000). Typically the amniotic fluid samples are processed to obtain preparations that are suitable for analysis by mass spectrometry. Such purification will usually include chromatography, such as liquid chromatography or capillary electrophoresis, and may also often involve an additional purification procedure that is performed prior to chromatography. Various procedures may be used for this purpose depending on the type of sample or the type of chromatography. Examples include filtration, centrifugation, combinations thereof and the like. The pH of the amniotic fluid sample may then be adjusted to any point required by a digestion agent. In some embodiments, the digestion agent is trypsin and pH can be adjusted with a solution of ammonium acetate to have a pH suitable for this enzyme. After trypsin digestion, the sample may be purified with a second filtration. The filtrate from this post-digestion filtration can then be purified by liquid chromatography and subsequently subjected to mass spectrometry analysis. Various methods have been described involving the use of high performance liquid chromatography (HPLC) for sample clean-up prior to mass spectrometry analysis. See, e.g., Taylor et al., Therapeutic Drug Monitoring 22:608-12 (2000) (manual precipitation of blood samples, followed by manual C18 solid phase extraction, injection into an HPLC for chromatography on a C18 analytical column, and MS/MS analysis); and Salm et al., Clin. Therapeutics 22 Supl. B:B71-B85 (2000). Commercially available HPLC columns include, but are not limited to, polar, ion exchange (both cation and anion), hydrophobic interaction, phenyl, C-2, C-8, C-18, and polar coating on porous polymer columns. During chromatography, the separation of materials is effected by variables such as choice of eluent (also known as a “mobile phase”), choice of gradient elution and the gradient conditions, temperature, etc. In some embodiments, the peptides are ionized by any method known to the skilled artisan. Mass spectrometry is performed using a mass spectrometer, which includes an ion source for ionizing the fractionated sample and creating charged molecules for further analysis. Ionization sources used in various MS techniques include, but are not limited to, electron ionization, chemical ionization, electrospray ionization (ESI), photon ionization, atmospheric pressure chemical ionization (APCI), photoionization, atmospheric pressure photoionization (APPI), fast atom bombardment (FAB)/liquid secondary ionization (LSIMS), matrix assisted laser desorption ionization (MALDI), field ionization, field desorption, thermospray/plasmaspray ionization, surface enhanced laser desorption ionization (SELDI), inductively coupled plasma (ICP) and particle beam ionization. The skilled artisan will understand that the choice of ionization method may be determined based on the analyte to be measured, type of sample, the type of detector, the choice of positive versus negative mode, etc. After the sample has been ionized, the positively charged ions thereby created may be analyzed to determine m/z. Suitable analyzers for determining m/z include quadrupole analyzers, ion trap analyzers, and time-of-flight analyzers. The ions may be detected using one of several detection modes. For example, only selected ions may be detected using a selective ion monitoring mode (SIM), or alternatively, multiple ions may be detected using a scanning mode, e.g., multiple reaction monitoring (MRM) or selected reaction monitoring (SRM). One may enhance the resolution of the MS technique by employing “tandem mass spectrometry,” or “MS/MS.” In this technique, a precursor ion (also called a parent ion) generated from a molecule of interest can be filtered in an MS instrument, and the precursor ion subsequently fragmented to yield one or more fragment ions (also called daughter ions or product ions) that are then analyzed in a second MS procedure. By careful selection of precursor ions, only ions produced by certain analytes are passed to the fragmentation chamber, where collision with atoms of an inert gas produce the fragment ions. Because both the precursor and fragment ions are produced in a reproducible fashion under a given set of ionization/fragmentation conditions, the MS/MS technique may provide an extremely powerful analytical tool. For example, the combination of filtration/fragmentation may be used to eliminate interfering substances, and may be particularly useful in complex samples, such as biological samples. Additionally, recent advances in technology, such as matrix-assisted laser desorption ionization coupled with time-of-flight analyzers (“MALDI-TOF”) permit the analysis of analytes at femtomole levels in very short ion pulses. Mass spectrometers that combine time-of-flight analyzers with tandem MS are also well known to the artisan. Additionally, multiple mass spectrometry steps may be combined in methods known as “MS/MS”. Various other combinations may be employed, such as MS/MS/TOF, MALDI/MS/MS/TOF, or SELDI/MS/MS/TOF mass spectrometry. One or more steps of the methods may be performed using automated machines. In some embodiments, one or more purification steps are performed on-line, and more preferably all of the LC purification and mass spectrometry steps may be performed in an on-line fashion.

In some embodiments, level of the peptide, protein, or protein fragment in the amniotic fluid sample is determined by is determined by CE-MS, in which capillary electrophoresis is coupled with mass spectrometry. This method has been described in some detail, for example, in the German Patent Application DE 10021737, in Kaiser et al. (J. Chromatogr A, 2003, Vol. 1013: 157-171, and Electrophoresis, 2004, 25: 2044-2055) and in Wittke et al. (J. Chromatogr. A, 2003, 1013: 173-181). The CE-MS technology allows to determine the presence of some hundreds of polypeptide markers of a sample simultaneously within a short time and in a small volume with high sensitivity. For CE-MS, the use of volatile solvents is preferred, and it is best to work under essentially salt-free conditions. Examples of suitable solvents include acetonitrile, methanol and the like. The solvents can be diluted with water or an acid (e.g., 0.1% to 1% formic acid) in order to protonate the analyte, preferably the polypeptides. By means of capillary electrophoresis, it is possible to separate molecules by their charge and size. Neutral particles will migrate at the speed of the electroosmotic flow upon application of a current, while cations are accelerated towards the cathode, and anions are delayed. The advantage of capillaries in electrophoresis resides in the favourable ratio of surface to volume, which enables a good dissipation of the Joule heat generated during the current flow. This in turn allows high voltages (usually up to 30 kV) to be applied and thus a high separating performance and short times of analysis. In capillary electrophoresis, silica glass capillaries having inner diameters of typically from 50 to 75 μm are usually employed. The lengths employed are 30-100 cm. In addition, the capillaries are usually made of plastic-coated silica glass. The capillaries may be either untreated, i.e., expose their hydrophilic groups on the interior surface, or coated on the interior surface. A hydrophobic coating may be used to improve the resolution. In addition to the voltage, a pressure may also be applied, which typically is within a range of from 0 to 1 psi. The pressure may also be applied only during the separation or altered meanwhile. Accordingly, in some embodiments, the markers of the sample are separated by capillary electrophoresis, then directly ionized and transferred on-line into a coupled mass spectrometer for detection.

Scores and Algorithms of the Present Invention:

In some embodiments, a score which is a composite of the expression levels of the different peptides is determined and compared to a reference value wherein a difference between said score and said reference value is indicative whether the fetus is at risk of having postnatal renal dysfunction. Typically, the predetermined reference value is a threshold value or a cut-off value, which can be determined experimentally, empirically, or theoretically. A threshold value can also be arbitrarily selected based upon the existing experimental and/or clinical conditions, as would be recognized by a person of ordinary skilled in the art. For example, retrospective measurement of the expression level of the selected peptide in properly banked historical amniotic samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the expression level of the selected peptide in a group of reference, one can use algorithmic analysis for the statistic treatment of the expression levels determined in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the method of the invention comprises the use of a classification algorithm typically selected from Linear Discriminant Analysis (LDA), Topological Data Analysis (TDA), Neural Networks, Support Vector Machine (SVM) algorithm and Random Forests algorithm (RF) such as described in the Example. In some embodiments, the method of the invention comprises the step of determining the subject response using a classification algorithm. As used herein, the term “classification algorithm” has its general meaning in the art and refers to classification and regression tree methods and multivariate classification well known in the art such as described in U.S. Pat. No. 8,126,690; WO2008/156617. As used herein, the term “support vector machine (SVM)” is a universal learning machine useful for pattern recognition, whose decision surface is parameterized by a set of support vectors and a set of corresponding weights, refers to a method of not separately processing, but simultaneously processing a plurality of variables. Thus, the support vector machine is useful as a statistical tool for classification. The support vector machine non-linearly maps its n-dimensional input space into a high dimensional feature space, and presents an optimal interface (optimal parting plane) between features. The support vector machine comprises two phases: a training phase and a testing phase. In the training phase, support vectors are produced, while estimation is performed according to a specific rule in the testing phase. In general, SVMs provide a model for use in classifying each of n subjects to two or more disease categories based on one k-dimensional vector (called a k-tuple) of biomarker measurements per subject. An SVM first transforms the k-tuples using a kernel function into a space of equal or higher dimension. The kernel function projects the data into a space where the categories can be better separated using hyperplanes than would be possible in the original data space. To determine the hyperplanes with which to discriminate between categories, a set of support vectors, which lie closest to the boundary between the disease categories, may be chosen. A hyperplane is then selected by known SVM techniques such that the distance between the support vectors and the hyperplane is maximal within the bounds of a cost function that penalizes incorrect predictions. This hyperplane is the one which optimally separates the data in terms of prediction (Vapnik, 1998 Statistical Learning Theory. New York: Wiley). Any new observation is then classified as belonging to any one of the categories of interest, based where the observation lies in relation to the hyperplane. When more than two categories are considered, the process is carried out pairwise for all of the categories and those results combined to create a rule to discriminate between all the categories. As used herein, the term “Random Forests algorithm” or “RF” has its general meaning in the art and refers to classification algorithm such as described in U.S. Pat. No. 8,126,690; WO2008/156617. Random Forest is a decision-tree-based classifier that is constructed using an algorithm originally developed by Leo Breiman (Breiman L, “Random forests,” Machine Learning 2001, 45:5-32). The classifier uses a large number of individual decision trees and decides the class by choosing the mode of the classes as determined by the individual trees. The individual trees are constructed using the following algorithm: (1) Assume that the number of cases in the training set is N, and that the number of variables in the classifier is M; (2) Select the number of input variables that will be used to determine the decision at a node of the tree; this number, m should be much less than M; (3) Choose a training set by choosing N samples from the training set with replacement; (4) For each node of the tree randomly select m of the M variables on which to base the decision at that node; (5) Calculate the best split based on these m variables in the training set. In some embodiments, the score is generated by a computer program.

In some embodiments, the method of the present invention comprises a) quantifying the level of a plurality of peptides of Table A in the amniotic sample; b) implementing a classification algorithm on data comprising the quantified plurality of peptides so as to obtain an algorithm output; c) determining the probability that the fetus will develop a postnatal renal dysfunction from the algorithm output of step b).

In some embodiments, the classification algorithm implements at least one clinical parameter. Typically said clinical parameter is selected from the group consisting of Age, gestational age at AF sampling; AF, amniotic fluid volume; bCAKUTPep-Age, combination of the bCAKUTPep classifier with gestational age at sampling; bCAKUTPep-AF, combination of the bCAKUTPep classifier with AF volume; bCAKUTPep-AF/Age, combination of the bCAKUTPep classifier with both gestational age at sampling and AF volume. In some embodiments, the method of the present invention further comprises determining the amniotic fluid volume (AF).

The algorithm of the present invention can be performed by one or more programmable processors executing one or more computer programs to perform functions by operating on input data and generating output. The algorithm can also be performed by, and apparatus can also be implemented as, special purpose logic circuitry, e.g., an FPGA (field programmable gate array) or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry. To provide for interaction with a user, embodiments of the invention can be implemented on a computer having a display device, e.g., in non-limiting examples, a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. Accordingly, in some embodiments, the algorithm can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet. The computing system can include clients and servers. A client and server are generally remote from each other and typically interact through a communication network. The relationship of client and server arises by virtue of computer programs running on the respective computers and having a client-server relationship to each other.

Kits or Devices of the Present Invention:

A further object of the present invention relates to a kit or device for performing the method of the present invention, comprising means for determining the level of the peptide or protein in the amniotic sample.

In some embodiments, the kit or device comprises at least one binding partner (e.g. antibody or aptamer) specific for the peptide or protein of interest (immobilized or not on a solid support as described above). In some embodiments, the kit or device can include a second binding partner (e.g. antibody or aptamer) of the present invention which produces a detectable signal. Examples of kits include but are not limited to ELISA assay kits, and kits comprising test strips and dipsticks.

In some embodiments, the kits or devices of the present invention further comprise at least one sample collection container for sample collection. Collection devices and container include but are not limited to syringes, lancets, BD VACUTAINER® blood collection tubes. In some embodiments, the kits or devices described herein further comprise instructions for using the kit or device and interpretation of results.

In some embodiments, the kit or device of the present invention further comprises a microprocessor to implement an algorithm on data comprising the plurality of peptides optionally with at least one clinical parameter (e.g. AF) in the sample so as to determine the probability of having a postnatal renal dysfunction for the fetus. In some embodiments, the kit or device of the present invention further comprises a visual display and/or audible signal that indicates the probability determined by the microprocessor.

In some embodiments, the kit or device of the present invention comprises:
a mass spectrometer;
a receptacle into which the amniotic fluid sample is placed, and which is connectable to the mass spectrometer so that the mass spectrometer can quantify the peptides in the sample;
a microprocessor to implement an algorithm on data comprising the plurality of peptides in the sample so as to determine the probability of having a postnatal renal dysfunction for the fetus;
a visual display and/or audible signal that indicates the probability determined by the microprocessor.

The invention will be further illustrated by the following figures and examples. However, these examples and figures should not be interpreted in any way as limiting the scope of the present invention.

### FIGURES

FIG. 1. Identification of amniotic fluid peptides predictive of postnatal renal function in bilateral CAKUT. Panel A shows the patients used in the training and in the blinded validation sets. Controls were defined as bilateral CAKUT fetuses with normal or moderately decreased renal function (eGFR>60 ml/min/m²) at two years of age, while cases were defined by early renal failure (e.g. eGFR<60 ml/min/m² at two years of age, or death due to end stage renal disease). Panel B displays the performance of the bCAKUTPep classifier based on the random forest mathematical combination of the 98 peptides in the training set. Left, ROC curve. Right, score of the bCAKUTPep classifier. The abscissa indicates the clinical end-point at 2 years. The dotted horizontal line indicates the cutoff score of 0.47 above which a patient is predicted to display severely altered postnatal renal function. Data are means plus or minus standard errors. ****P<0.0001, Mann-Whitney test for independent samples. Confidence intervals, given in brackets, for the AUC, sensitivity and specificity are two-sided 95% CI.

FIG. 2. Validation of the amniotic fluid peptide based classifier and comparison to clinical parameters. Panel A shows the performance of the amniotic fluid peptide based classifier, bCAKUTPep, in the validation cohort composed of 51 patients with bilateral CAKUT (34 controls and 17 cases). Left, ROC curve. Right, scores of the bCAKUTPep classifier in the validation set. The dotted horizontal line indicates the cutoff score of 0.47 above which a patient is predicted to display severely altered postnatal renal function. The abscissa indicates the clinical end-point at 2 years. Data are means plus or minus standard errors. ****P<0.0001, Mann-Whitney test for independent samples. Panel B shows the ROC curve of the bCAKUTPep classifier compared to clinical parameters or to its combination with those clinical parameters in the validation set. Age, gestational age at AF sampling; AF, amniotic fluid volume; bCAKUTPep-Age, combination of the bCAKUTPep classifier with gestational age at sampling; bCAKUTPep-AF, combination of the bCAKUTPep classifier with AF volume; bCAKUTPep-AF/Age, combination of the bCAKUTPep classifier with both gestational age at sampling and AF volume. Panel C shows the ROC curves in the validation set of the 98 peptides combined in different mathematical models. SVM, a support vector machine model; Linear, a linear model; KNN, a k-nearest neighbors model. Panel D shows the ROC curves for the geographical validation of the bCAKUTPep classifier. All patients, all patients in the validation set; Belgium patients, 12 patients from the validation set with a distinct geographical origin; All—Belgium patients, the validation set without the Belgium patients. Panel E shows the domain validation using 22 healthy fetuses from pregnancies and 47 fetuses with primary maternal CMV infection¹¹. The dotted horizontal line indicates the cutoff score of 0.47 above which a patient is predicted to display severely altered postnatal renal function. Confidence intervals given in brackets for AUC, sensitivity and specificity are two-sided 95% CI except in panel B where they are upper limit of the one-sided 95% CI.

FIG. 3. Use of the peptide-based classifier in specific CAKUT scenarios. Panel A shows the prediction of postnatal renal function by the bCAKUTPep classifier of 8 termination of pregnancies (TOPs) in bilateral CAKUT pregnancies where fetopathology, analysed by three independent pathologists, displayed a renal phenotype type that appeared compatible with normal life. Panel B shows the prediction of postnatal renal function by the bCAKUTPep classifier of 28 TOPs in CAKUT pregnancies where fetopathology was inconclusive or not available. A bCAKUTPep value above the 0.47 cutoff suggests severely altered postnatal renal function.

### EXAMPLE

Methods

Study Patients

Two-hundred women consented to participate in the study, including 178 originally identified as having a pregnancy with a fetus presenting bilateral CAKUT (data not shown) and 22 from non-CAKUT pregnancies. The 22 samples from non-CAKUT fetuses were obtained from pregnancies tested, but being negative, for chromosomal abnormalities. During follow-up of the 178 CAKUT patients, 28 pregnancies were excluded. The trial was performed in accordance with the Declaration of Helsinki and with Good Clinical Practice guidelines. Patients were recruited in France and in Belgium. For all patients definite information on the renal status after 2 years of postnatal follow-up was obtained. The research was approved by national ethics committees (No RCB 2010-AO1151-38, France and S 55406 and B32220096569, Belgium) and informed consent was obtained from each participant.

Fetopathology and Analysis of Renal Function

Fetopathology was assessed for fetuses after termination of pregnancy (TOP) by 3 independent pathologists and were attributed a severity renal score: HS, high severity, defined by extensive dysplasia and/or hypoplasia; S, severe, segmental dysplasia and/or hypoplasia with alternation between healthy and pathological areas; LS, low severity, corresponding to kidneys with nearly normal parenchyma or little segmental dysplasia and/or hypoplasia. Dysplasia was defined by alteration of the renal structure with both glomerular and tubular lesions, persistence of primitive medullar tubules surrounded by fibromuscular cells and cartilaginous islets; hypoplasia was histologically defined by a reduction of structurally normal nephron number. At least one HS score without any LS score was interpreted as fetuses with renal lesions incompatible with normal life. At least two LS scores without any HS score was interpreted as compatible with normal life. All other combinations of scores or absence of fetopathology data were considered as inconclusive. Renal function was estimated at 2 years of life using serum creatinine concentrations according the Schwartz method¹³.

Sample Collection and Preparation, Peptidome Analysis and Data Processing

AF collection, sample preparation, and peptidome analysis by capillary electrophoresis coupled to mass spectrometry (CE-MS) and data processing were previously described¹².

Statistical Analysis

Significant peptides were selected by Wilcoxon analysis followed by correction for multiple testing using the method of Benjamini-Hochberg¹⁴. The prognostic ‘bCAKUTPep’ peptide classifier was generated using the Random Forest (RF)-package¹⁵ of R. Predictive performance was assessed by calculating sensitivity, specificity, area under the receiver-operating-characteristic curve (AUC) and likelihood ratios using Medcalc (Version 14.12.0).

Results:

Characteristics of the Study Population

Among the 140 prospectively included patients with bilateral CAKUT, the major etiologies were hyperechogenic kidneys (40/140) and lower urinary tract obstruction (29/140) representing 49% of the patients (Table 1).

69/140 (49%) of the fetuses had normal or moderately reduced renal function (eGFR>60 ml/min/1.73 m²) at 2 years postnatally. Etiologies mostly associated to normal outcome were non-obstructive urinary tract anomalies and upper urinary tract obstruction. In contrast, 71/140 (51%) of the fetuses developed postnatal CKD (eGFR<60 ml/min/1.73 m² at 2y) or perinatal death due to ESRD or were subjected to termination of pregnancy (TOP). Non-functioning kidneys and lower urinary tract obstruction were the main etiologies associated to these poor outcomes.

Severe renal lesions incompatible with CKD-free survival were confirmed by fetopathology for 24 of the 60 fetuses submitted to TOP. Considering only patients for which we had definite endpoint data, the prevalence of early renal failure was 33% in the bilateral CAKUT population.

Identification of Predictive Amniotic Fluid Peptides

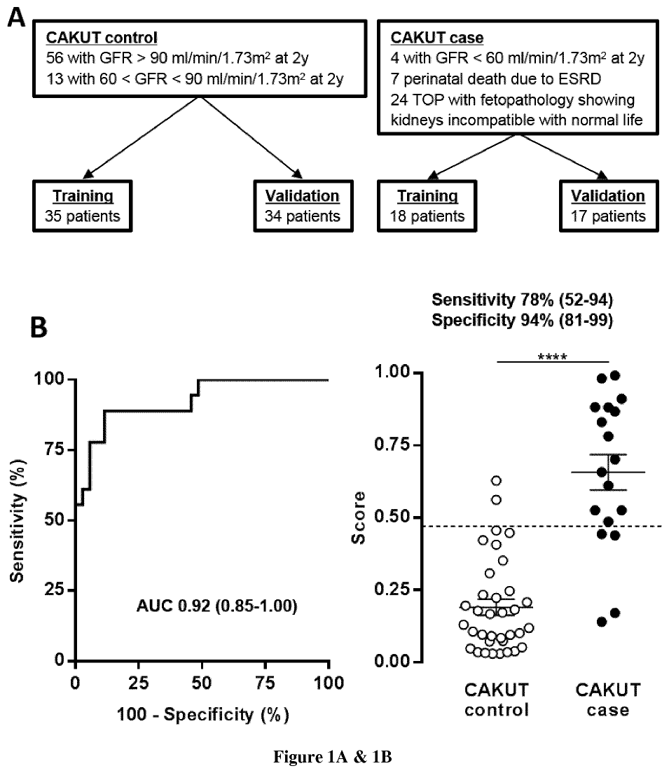

The prospective cohort of 140 bilateral CAKUT fetuses was divided in independent training and validation sets (FIG. 1A and data not shown). The training set included 35 CAKUT with normal or moderately reduced renal function (eGFR>60 ml/min/1.73 m² at age 2 years) defined as “CAKUT control” and 18 CAKUT with compromised outcome (2-year eGFR<60 ml/min/1.73 m², perinatal death due to ESRD, or TOP with fetopathology showing severe renal maldevelopment) defined as “CAKUT case” (data not shown). A total of 7,000 peptides were detected in AF, for 1,008 of which sequence information could be obtained. Comparison of CAKUT case versus CAKUT control yielded 98 peptides with significantly different abundance (corrected p-values) and multi-fold changes (up to 100 fold) (Table A). The majority of the peptides were fragments of various collagens (88%). Other peptides included fragments of thymosin-β4 (3%), inter a trypsin inhibitor heavy chain H4 (2%) and fibrinogen a chain (2%) (data not shown). Increased abundance of a thymosin β4 fragment was confirmed using an enzyme-linked immunosorbent assay in a subset of patients (data not shown).

The 98 peptides were included in a random forest mathematical model (called the ‘bCAKUTPep’ classifier), which was optimized for the classification of patients in the training cohort. Based on a cutoff score of 0.47, the bCAKUTPep classifier led to a prediction of postnatal renal outcome with a sensitivity of 78%, a specificity of 94% and an AUC of 0.92 (FIG. 1B).

Validation of the Peptide-Based Classifier in New Individuals

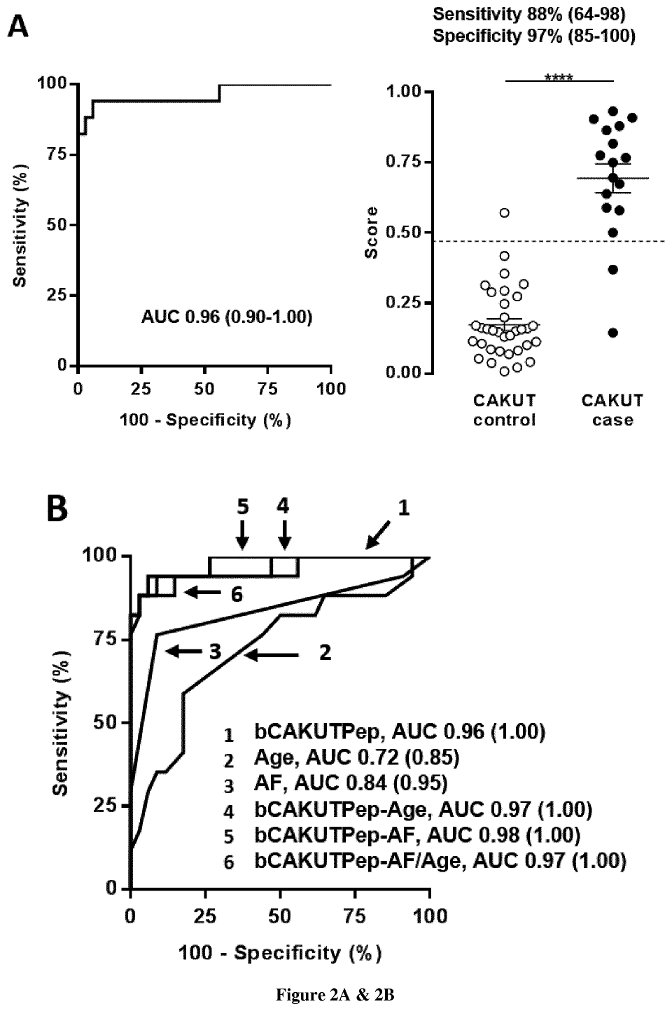

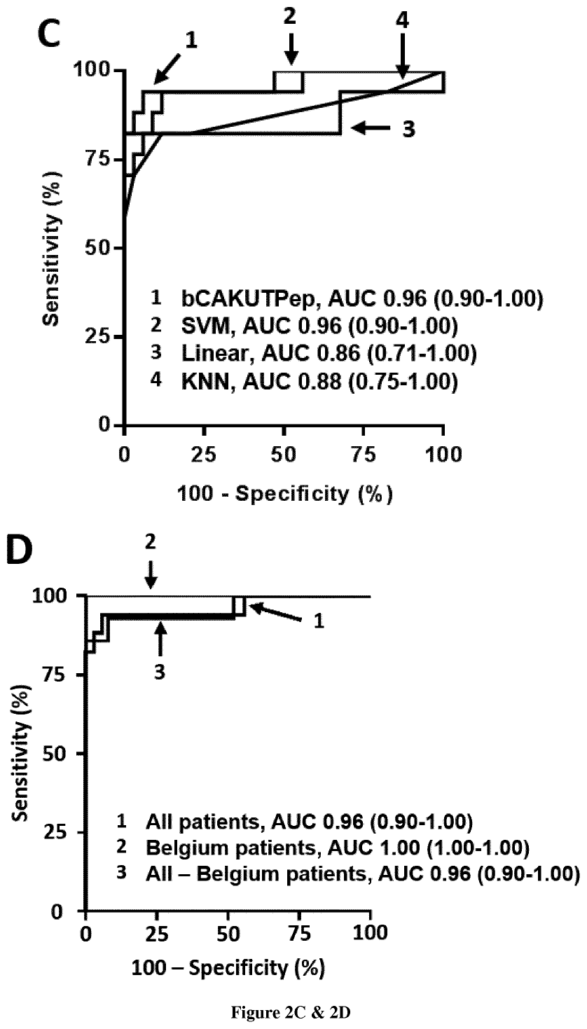

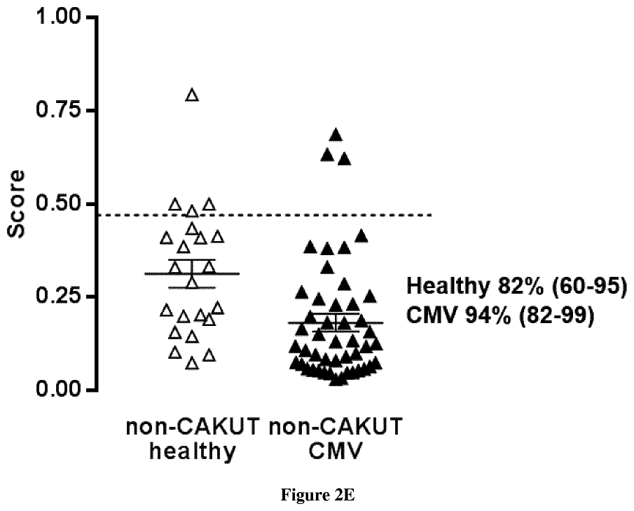

It is essential to confirm that predictive biomarkers are generalizable to ‘similar but different’ individuals outside the training set^{16,17}. Therefore in the next step we blindly validated bCAKUTPep in the hold out validation set of 51 patients composed of 34 additional CAKUT control and 17 CAKUT case patients (data not shown). This resulted in prediction of postnatal renal function with 88% sensitivity, 97% specificity, an AUC of 0.96 (FIG. 2A), and positive and negative likelihood ratios of 30 and 0.12, respectively.

Comparison with Clinical Parameters

The predictive efficacy of the bCAKUTPep classifier was next compared to the clinical parameters including routinely performed ultrasound-based clinical measurements and gestational age at the time of AF sampling. The presence of hyperechogenicity, absence of corticomedullary differentiation (dysplasia), or at least one nonfunctional kidney (MCDK or agenesis) failed to predict postnatal renal function (AUC: 0.60, 0.60 and 0.54, respectively, data not shown). Reduced AF volume (oligohydramnios or anhydramnios) or gestational age at sampling predicted postnatal renal outcome with 76% sensitivity and 91% specificity (AUC: 0.84) and 59% sensitivity and 82% specificity (AUC: 0.72), respectively. Both parameters were significantly inferior compared to the peptide-based classifier (FIG. 2B and data not shown).

We next assessed whether the predictive performance of the peptide-based classifier could be improved by adding the clinical parameters showing the best individual performances. Combination of the peptides with either AF volume or gestational age, or both showed a slightly, but non-significant, increase in AUC (0.98, 0.97 and 0.97, respectively) compared to bCAKUTPep alone (0.96, FIG. 2B and data not shown).

Robustness of the Peptide-Based Classifier

The 98 selected peptides behaved similarly well when using other mathematical approaches including support vector machine (SVM), a k-nearest neighbor (KNN) or linear models (FIG. 2C and data not shown) suggesting the robustness of the 98 biomarker peptides. Furthermore, geographical validation of the classifier using a small subset of patients from the validation cohort, i.e. 12 patients with CAKUT from Belgium (Belgium was not included in the training phase), showed excellent prediction (AUC: 1.00, FIG. 2D and data not shown). Finally we performed a domain validation of the classifier to test its specificity in individuals having a very different clinical status than CAKUT (22 healthy fetuses from pregnancies of healthy women and 47 fetuses with primary maternal CMV infection¹²). The bCAKUTPep classifier predicted normal postnatal renal function with a specificity of 82% and 94% in the two cohorts, respectively (FIG. 2E and data not shown). This combined evidence supports the robustness and wider applicability of the AF peptide-based classifier.

Application of the Peptide-Based Classifier in Specific CAKUT Scenarios

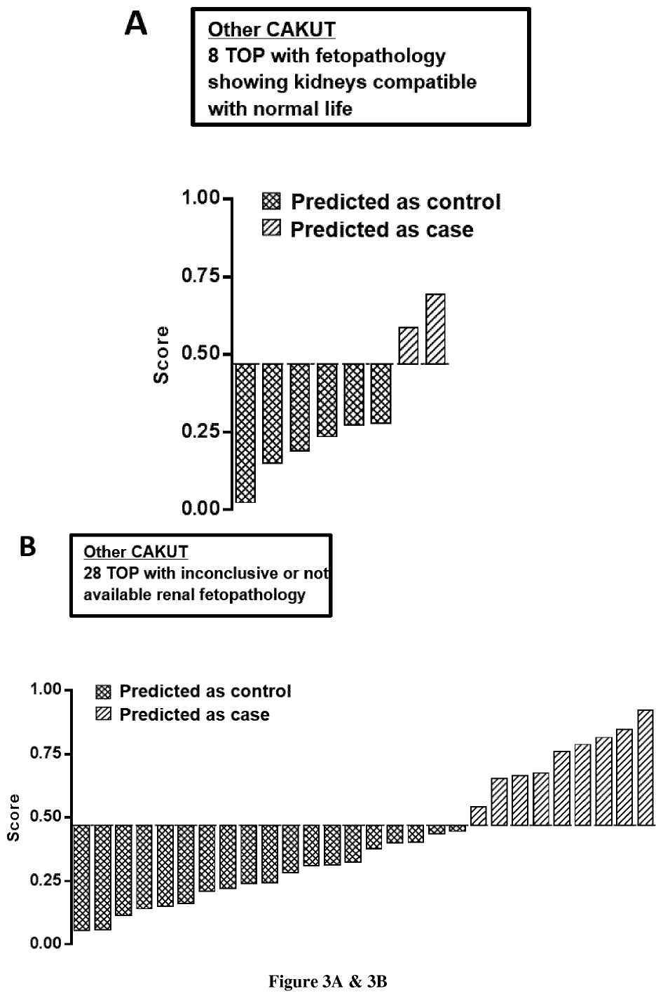

Among the 32 bilateral CAKUT pregnancies submitted to TOP for which we had definitive fetopathology description, 8 fetuses displayed a renal phenotype that appeared compatible with life (Table S5) in Supplementary Appendix). bCAKUTPep predicted normal postnatal renal function for 6 of them, thereby confirming fetopathology (FIG. 3A).

For 28 of the 60, fetopathology was inconclusive or not available (data not shown). The bCAKUTPep classifier predicted early renal failure for 9 patients while normal postnatal renal function was predicted for 19 patients (FIG. 3B).

Selection of Smallest Predictive Peptide Signatures

Signatures including one peptide (clusters 1P): The ability of each peptide reported in Table A to predict postnatal renal outcome in bilateral CAKUT was evaluated measuring AUC of the ROC curve from the validation set. A peptide was judged excellent when it was better in prediction than AF volume, a clinical routinely measured parameter. Considering that AUC>=0.95 was significantly superior to AUC of AF volume (0.84 [upper limit of the one-sided 95% CI: 0.95]), one peptide (ID: 31862) was selected (Table 2).

Signatures including two peptides (clusters 2P): mathematical models (random forest or support vector machine) combining together 2 peptides reported in Table A (except the peptide included in the Table 2) were developed. After optimization for the classification of patients in the training set, models were assessed for the prediction of postnatal renal outcome in bilateral CAKUT measuring AUC of the ROC curve from the validation set. A cluster 2P was judged excellent when it was better in prediction than AF volume, a clinical routinely measured parameter. Considering that AUC>=0.95 was significantly superior to AUC of AF volume (0.84 [upper limit of the one-sided 95% CI: 0.95]), 38 clusters 2P involving a total of 35 peptides were selected (Table 3).

Signatures including three peptides (clusters 3P): mathematical models (random forest or support vector machine) combining together 3 peptides reported in Table A (except the peptides included in both Tables 2-3) were developed. After optimization for the classification of patients in the training set, models were assessed for the prediction of postnatal renal outcome in bilateral CAKUT measuring AUC of the ROC curve from the validation set. A cluster 3P was judged excellent when it was better in prediction than AF volume, a clinical routinely measured parameter. Considering that AUC>=0.95 was significantly superior to AUC of AF volume (0.84 [upper limit of the one-sided 95% CI: 0.95]), 77 clusters 3P involving a total of 40 peptides were selected (Table 4).

Selection of Smallest Predictive Peptide Signatures Associated to Amniotic Fluid Volume

Signatures including one peptide and AF volume (clusters 1P+AF): each peptide reported in Table A was included with AF volume, a clinical routinely measured parameter, in mathematical models (random forest or support vector machine) which were optimized for the classification of patients in the training set. The efficacy of each cluster to predict the postnatal renal outcome in bilateral CAKUT was evaluated measuring AUC of the ROC curve from the validation set. A cluster 1P+AF was judged excellent when it was better in prediction than AF volume. Considering that AUC>=0.95 was significantly superior to AUC of AF volume (0.84 [upper limit of the one-sided 95% CI: 0.95]), 9 clusters 1P+AF thereby corresponding to 9 peptides were selected (Table 5).

Signatures including two peptides and AF volume (clusters 2P+AF): mathematical models (random forest or support vector machine) combining together 2 peptides reported in Table A (except the peptides included in the Table 5) as well as AF volume were developed. After optimization for the classification of patients in the training set, models were assessed for the prediction of the postnatal renal outcome in bilateral CAKUT by measuring AUC of the ROC curve from the validation set. A cluster 2P+AF was judged excellent when it was better in prediction than AF volume, a clinical routinely measured parameter. Considering that AUC>=0.95 was significantly superior to AUC of AF volume (0.84 [upper limit of the one-sided 95% CI: 0.95]), 865 clusters 2P+AF involving 86 peptides were selected (Table 6).

Thymosin-β4 Protein-Based Prediction

Thymosin-β4 alone: Quantification of thymosin-β4 was performed by meaning the abundance of its 3 fragments (peptide ID: 31862, 35677, 33930, reported in Table A). The ability of protein to predict postnatal renal outcome in bilateral CAKUT was evaluated measuring AUC of the ROC curve from the validation set. Compared to AF volume, thymosin-β4 showed an increasing trend in AUC, but without reaching statistical significance (0.94 versus 0.84, p=0.066) (Table 7).

Thymosin-β4 combined to AF volume (Thymosin-/34+AF): Quantification of thymosin-β4 was performed by meaning the abundance of its 3 fragments (peptide ID: 31862, 35677, 33930, reported in Table A). Thymosin-β4 was included with AF volume in a random forest model which was optimized for the classification of patients in the training set. The efficacy of Thymosin-β4+AF to predict the postnatal renal outcome in bilateral CAKUT was evaluated measuring AUC of the ROC curve from the validation set. Compared to AF volume alone, thymosin-β4+AF displayed a significant increase in AUC (0.95 versus 0.84; one-sided p value: 0.040) (data not shown).

Ac-SDPK Fragment-Based Prediction

N-acetyl-seryl-aspartyl-lysyl-proline (Ac-SDKP), a natural tetrapeptide released from thymosin-β4, was measured in amniotic fluid from a subset of patients using an enzyme-linked immunosorbent assay. Ac-SDKP was included with AF volume (Ac-SDKP+AF) in a support vector machine model and the efficacy of the model to predict the postnatal renal outcome in bilateral CAKUT was evaluated measuring AUC of the ROC curve in the same subset. Ac-SDKP+AF displayed a significant increase in AUC compared to AF volume alone (0.98 versus 0.86; one-sided p value: 0.042) (Table 8).

Discussion:

Unambiguous prenatal prediction of postnatal renal function in bilateral CAKUT, not attainable by conventional clinical and imaging parameters, would provide an evidence base for rational and ethically sound management of this challenging disorder. Using samples from the largest prospective bilateral CAKUT cohort followed to date, we developed and blindly validated a novel method for the prediction of postnatal renal function based on the analysis of peptides in amniotic fluid. Based on a numerical score with a clear-cut cutoff, the AF peptide-based classifier (bCAKUTPep) predicted postnatal renal function with high sensitivity and specificity, significantly outperforming ultrasound measures. Hence, the AF peptide-based classifier is an innovative methodology with disruptive potential for the pre- and postnatal management of bilateral CAKUT.

Counseling of parents-to-be with a fetus with bilateral CAKUT is emotion loaded as it often involves the consideration to terminate pregnancy in the face of a highly uncertain prognosis ranging from largely normal postnatal kidney function to perinatal death or life-long end-stage kidney disease. The AF peptide signature established in this study provides for the first time an unambiguous prediction of postnatal kidney function with much higher accuracy compared to conventional methods. In addition, the measurement of AF peptides is not subject to personal interpretation, which can be the case for sonographic imaging^{6,18 19} (e.g. an obstetrician versus pediatric nephrologist/urologist, a less versus a more experienced clinician). Hence, the AF peptide score provides unbiased information concerning the likely postnatal outcome to the parents²⁰. In addition, in case a high-risk phenotype is diagnosed and continuation of pregnancy is decided, such clear-cut information will also give time to the future parents to psychologically accept²¹ the fact that they will have a child with chronic, potentially severe disease and decide whether they would like their newborn to be offered palliative care or dialysis²².

In our large scale prospective study 60 out of 140 (43%) CAKUT pregnancies were terminated. This rate is slightly lower than in previous European studies where the rate of pregnancy termination was 55-62%^{8,23-25}, but close to a recent retrospective study from the US (45% (32/71))²⁶. Irrespective of these differences, termination of pregnancy is still a major decision in CAKUT fetuses and in a number of cases, as exemplified by our study, fetopathology analysis revealed fetal kidneys with normal appearance. The added value of the AF peptide-based classifier in this context is evident from the fact that bCAKUTPep predicted a normal outcome for 6 out of the 8 terminated pregnancies in which fetopathology showed kidneys that appeared compatible with normal life. In 28 cases of pregnancy termination where fetopathology was absent (usually due to parental non-consent) or inconclusive (no definite status as to the severity of the renal lesions), the bCAKUTPep classifier predicted 9 fetuses (32%) with a severe outcome. This is very similar to the number of severe outcomes (34%) in our cohort for whom we had definitive outcome data, thereby confirming the high positive predictive value of the AF peptide classifier. Therefore, in case of absent or inconclusive fetopathology (nearly 50% of the terminated pregnancy cases studied) a severe AF peptide score might alleviate the psychological burden imposed on the parents after the decision to terminate pregnancy.

Postnatal events or interventions (e.g. urinary tract infections or obstruction-relieving interventions), can impact postnatal disease progression. However, among the 17 fetuses with severe disease in the validation set, twelve were terminated pregnancies and three deceased perinatally. Of the 2 life-born children, one had bilaterally enlarged hyperechogenic dysplastic microcystic kidneys without urinary tract anomalies, and the other had PUV but was free of urinary tract infections during follow-up. In addition, potentially outcome-changing prenatal interventions such as vesico-amniotic shunting in PUV were not performed in our study³⁰.

We recently observed that the presence of specific urinary collagen peptides is related to the degree of in situ kidney fibrosis in adult CKD²⁷ and that these peptides are predictive of disease progression²⁸. Similarly, we speculate that a focus on the AF peptides may allow assessing the early underlying molecular changes of CAKUT such as connective tissue turnover (collagen fragments) leading to hypo/dysplasia and hyperechogenicity, inflammation (osteopontin, inter a trypsin inhibitor heavy chain H4) and repair (thymosin β4). As these early molecular modifications precede structural and functional changes, this may explain the excellent predictive capacity of the AF peptide signature as to postnatal renal function.

A limitation of our study is that we have not compared the AF peptides to the performance of serum β2-microglobulin. This would have required an additional invasive intervention for collecting fetal blood in our study while evidence in the literature for good predictive performance of serum β2-microglobulin is still lacking^{8,9}. However comparison with published sensitivities and specificities showed that the AF peptide-based classifier performed much better than fetal serum β2-microglobulin, at least in the context of bilateral lower or upper urinary tract obstruction⁸ (64% sensitivity and 79% specificity for β2-microglobulin⁸ versus 86% sensitivity and 100% specificity for bCAKUTPep).

Another limitation might be that the analysis is mass spectrometry-based since it is currently impossible to simultaneously analyze 98 peptides using an antibody-based method. However, we have shown in previous studies that samples can be frozen in the clinic, shipped and analyzed in specialized laboratories equipped with CE-MS technology^{11,29} with a total turnaround time of less than one week, an acceptable timeframe for clinical decision-making in CAKUT pregnancies.

In conclusion, we firmly believe that the introduction of the bCAKUTPep classifier in the diagnostic workup of prenatally diagnosed CAKUT can provide a long-sought evidence base to the prenatal counseling process by delivering unbiased and unambiguous prognostic information that is currently unavailable.

Tables:

TABLE 1
Antenatal cohort characteristics of 140 CAKUT patients of which the amniotic fluid peptidome was analyzed.

| | | | Gestational | Amniotic | | Outcome^{γ} | |
|---|---|---|---|---|---|---|---|
| | | Gender | age | fluid | | GFR < 60 | |
| | N | (f/m)¶ | (w)¥ | (n.a, n, o, a)§ | GFR > 60 | or death | TOP |
| Total cases | 140 | 40/82 | 25.68 +/− 0.50 | 18, 68, 42, 12 | 69 | 11 | 60 |
| Bilateral hyperechogenic kidneys | | | | | | | |
| normal size | 17 | 4/12 | 26.06 +/− 1.29 | 2, 12, 3, 0 | 10 | 0 | 7 |
| enlarged | 23 | 14/8 | 27.35 +/− 1.30 | 4, 12, 7, 0 | 10 | 2 | 11 |
| Lower urinary tract obstruction | | | | | | | |
| PUV | 25 | 0/20 | 24.64 +/− 1.40 | 3, 3, 16, 3 | 5 | 4 | 16 |
| others | 4 | 1/2 | 17.50 +/− 1.19 | 1, 3, 0, 0 | 1 | 0 | 3 |
| Abnormal solitary kidney* | | | | | | | |
| agenesis | 9 | 2/7 | 27.44 +/− 1.40 | 3, 2, 3, 1 | 4 | 1 | 4 |
| MCDK | 12 | 6/5 | 28.17 +/− 1.11 | 1, 8, 3, 0 | 8 | 1 | 3 |
| Upper urinary tract obstruction** | 17 | 1/11 | 25.12 +/− 1.15 | 2, 14, 1, 0 | 15 | 1 | 1 |
| Bilateral hypoplasia | 10 | 4/6 | 25.00 +/− 1.56 | 1, 4, 4, 1 | 5 | 0 | 5 |
| Nonfunctioning kidneys*** | 8 | 3/5 | 20.75 +/− 1.77 | 0, 0, 1, 7 | 0 | 0 | 8 |
| Non obstructive urinary tract anomalies**** | 7 | 2/1 | 23.43 +/− 2.16 | 0, 6, 1, 0 | 7 | 0 | 0 |
| Bilateral dysplasia | 5 | 1/4 | 28.80 +/− 2.82 | 1, 3, 1, 0 | 3 | 1 | 1 |
| One hypoplastic and one dysplastic kidney | 3 | 2/1 | 33.67 +/− 2.73 | 0, 1, 2, 0 | 1 | 1 | 1 |

*One nonfunctional (agenesis or multicystic dysplastic kidney (MCDK)) kidney and one kidney with either ureteropelvic junction obstruction (UPJ) with parenchymal lesions or dysplasia or hypoplasia or hyperechogenecity or combinations thereof;
**Bilateral UPJ with bilateral parenchymal lesions;
***Bilateral agenesis or MCDK;
****Vesicoureteral reflux, duplex collecting system, megaureter;
¶Gender of fetus, female/male (18 missing values);
¥Gestational age plus or minus standard error in weeks;
§Amniotic fluid volume: n.a, not available; n, normal; o, oligoamnios; a, anhydramnios;
^{γ}Post natal pregnancy outcome at two years: GFR > 60, normal renal function or moderately reduced renal function (eGFR > 60 ml/min/1.73 m²);
GFR < 60 or death, eGFR < 60 ml/min/1.73 m² or death due to renal dysfunction;
TOP, termination of pregnancy;
Abbreviation: PUV, posterior urethral valves.

TABLE A
List of 98 peptides associated to CAKUT progression.

| | | | | | | | |
|---|---|---|---|---|---|---|---|
| Peptide ID | 0.0342 | 0.0162 | 0.0375 | 0.02 | 0.0124 | 0.025 | 0.02 |
| Mass (Da) | UP | UP | UP | DOWN | DOWN | DOWN | DOWN |
| calc. Mass (Da) | 14.68 | 20.45 | 1.92 | 0.37 | 0.44 | 0.49 | 0.53 |
| CE-time (min) | 102.33 | 338.92 | 12.28 | 11.06 | 39.19 | 12.24 | 201.11 |
| Sequence | 6.97 | 16.57 | 6.41 | 29.58 | 88.17 | 25.01 | 378.97 |
| Protein name | H7C0L5 | H7C0L5 | P02452 | P02452 | P02452 | P02452 | P02452 |
| Start AA | 510 | 511 | 784 | 1071 | 725 | 455 | 843 |
| Stop AA | 501 | 499 | 766 | 1042 | 699 | 430 | 819 |
| Protein | Inter- | Inter- | Collagen | Collagen | Collagen | Collagen | Collagen |
| Accession | alpha- | alpha- | alpha- | alpha- | alpha- | alpha- | alpha- |
| | trypsin | trypsin | 1(1) | 1(1) | 1(1) | 1(1) | 1(1) |
| | inhibitor | inhibitor | chain | chain | chain | chain | chain |
| | [symbol] | [symbol] | | | | | |
| CAKUT | PGPPDV | GLPGP | TGPIGP | AGPGAGA | ANGAG | KGNSGE | ADGQP |
| control | PDHA | PDVPD | GPAGA | PGAPGVG | NDGAK | PGAGS | GAKGE |
| abundance | (SEQ ID | HAA | GDKGES | PAGKSGDR | GDAGA | KGDTGA | GDAGA |
| | NO: | (SEQ ID | (SEQ | GETGP | GAGSQ | KGEGP | KGDAG |
| | 1) | NO: 2) | ID NO: 3) | (SEQ | GAG | VG | PGP |
| | | | | ID NO: 4) | (SEQ ID | (SEQ ID | (SEQ ID |
| | | | | | NO: 5) | NO: 6) | NO: 7) |
| CAKUT ease abundance | 26.565176 | 27.284883 | 30.896709 | 28.233681 | 33.505863 | 22.767244 | 26.843721 |
| Fold change | 1000.46141 | 1241.604051 | 1692.795494 | 2583.231357 | 2281.975133 | 2339.098946 | 2204.993418 |
| Regulation | 1000.474548 | 1241.570313 | 1692.774902 | 2583.199219 | 2281.983398 | 2339.0896 | 2204.994141 |
| Ajusted p-value | 2029 | 6400 | 15884 | 29894 | 25170 | 26070 | 23894 |
| Peptide | 0.032 | 0.0054 | 0.0084 | 0.0073 | 0.0039 | 0.0233 | 0.0027 |
| ID | | | | | | | |
| Mass | DOWN | DOWN | DOWN | DOWN | DOWN | UP | DOWN |
| (Da) | | | | | | | |
| calc. | 0.56 | 0.36 | 0.41 | 0.4 | 0.27 | 2.06 | 0.3 |
| Mass | | | | | | | |
| (Da) | | | | | | | |
| CE-time | 108.05 | 5.84 | 16.12 | 4.82 | 1.02 | 20.49 | 14.03 |
| (min) | | | | | | | |
| Sequence | 192.47 | 16.4 | 39.21 | 11.93 | 3.83 | 9.95 | 46.46 |
| Protein | P02452 | P02452 | P02452 | P02452 | P02452 | P02452 | P02452 |
| name | | | | | | | |
| Start AA | 1041 | 843 | 844 | 668 | 453 | 843 | 844 |
| Stop AA | 1010 | 819 | 819 | 650 | 432 | 820 | 819 |
| Protein Accession | Collagen | Collagen | Collagen | Collagen | Collagen | Collagen | Collagen |
| | alpha- | alpha- | alpha- | alpha- | alpha- | alpha- | alpha- |
| | 1(I) chain | 1(I) chain | 1(I) chain | 1(I) chain | 1(I) chain | 1(I) chain | 1(I) chain |
| CAKUT | GESGRE | ADGQGAKGEG | ADGQGAKGEG | GPGEAGKGEQG | NSGEPGAGSKG | DGQPGAKGEPG | ADGQPGAKGE |
| control | GAGAE | DAGAKGDAGG | DAGAKGDAGP | VGDLG (SEQ | DTGAKGEGP | DAGAKGDAGPP | GDAGAKGDAG |
| abundance | GSPGRD | P (SEQ ID NO: | GPA (SEQ ID | ID NO: 11) | (SEQ ID NO: | G (SEQ ID NO: | PGPA (SEQ ID |
| | GSGAK | 9) | NO: 10) | | 12) | 13) | NO: 14) |
| | GDRGET | | | | | | |
| | G | | | | | | |
| | (SEQ ID | | | | | | |
| | NO: 8) | | | | | | |
| CAKUT | 22.135977 | 26.984415 | 27.169418 | 31.024588 | 25.256365 | 27.232393 | 27.162773 |
| ease | | | | | | | |
| abundance | | | | | | | |
| Fold | 2999.320125 | 2236.983248 | 2292.025447 | 1765.811872 | 1997.892642 | 2117.96139 | 2276.030532 |
| change | | | | | | | |
| Regulation | 2999.301758 | 2236.985352 | 2292.017578 | 1765.781616 | 1997.903931 | 2117.951416 | 2276.014404 |
| Ajusted | 36283 | 24421 | 25301 | 17264 | 20643 | 22456 | 25060 |
| p-value | | | | | | | |
| Peptide | 0.0159 | 0.0058 | 0.0124 | 0.0014 | 0.0142 | 0.0025 | 0.0152 |
| ID | | | | | | | |
| Mass | DOWN | DOWN | DOWN | DOWN | DOWN | DOWN | DOWN |
| (Da) | | | | | | | |
| calc. | 0.51 | 0.37 | 0.6 | 0.46 | 0.37 | 0.25 | 0.54 |
| Mass | | | | | | | |
| (Da) | | | | | | | |
| CE-time (min) | 220.89 | 30.99 | 8.81 | 71.53 | 2.18 | 1.48 | 24.4 |
| Sequence | 436.2 | 83.56 | 14.74 | 156.08 | 5.94 | 6.01 | 45.16 |
| Protein name | P02452 | P02452 | P02452 | P02452 | P02452 | P02452 | P02452 |
| Start AA | 1041 | 558 | 453 | 843 | 249 | 453 | 453 |
| Stop AA | 1007 | 543 | 433 | 819 | 232 | 432 | 431 |
| Protein | Collagen alpha- | Collagen alpha- | Collagen alpha- | Collagen alpha- | Collagen alpha- | Collagen alpha- | Collagen alpha- |
| Accession | 1(I) chain | 1(I) chain | 1(I) chain | 1(I) chain | 1(I) chain | 1(I) chain | 1(I) chain |
| CAKUT control | GPGESGREGAG | SGSGPDGKTGP | SGEGAGSKGDT | ADGQGAKGEG | DGEAGKPGRGE | NSGEGAGSKGD | GN SGEG AGSKG |
| abundance | AEGSGRDGSPG | GP (SEQ ID | GAKGEGP (SEQ | DAGAKGDAGP | RGPPG (SEQ ID | TGAKGEG | DTGAKGEGP |
| | AKGDRGETG | NO: 16) | ID NO: 17) | GP (SEQ ID | NO: 19) | (SEQ ID NO: | (SEQ ID NO: |
| | (SEQ ID NO: | | | NO: 18) | | 20) | 21) |
| | 15) | | | | | | |
| CAKUT | 22.470945 | 29.067095 | 24.954149 | 26.930149 | 21.495632 | 25.364849 | 25.570875 |
| ease | | | | | | | |
| abundance | | | | | | | |
| Fold | 3266.442031 | 1451.652852 | 1899.844629 | 2220.988333 | 1761.839424 | 2029.882471 | 2070.90902 |
| change | | | | | | | |
| Regulation | 3266.443848 | 1451.652344 | 1899.85791 | 2220.989502 | 1761.844971 | 2029.893799 | 2070.918213 |
| Ajusted | 40022 | 11078 | 19221 | 24148 | 17207 | 21076 | 21684 |
| p-value | | | | | | | |
| Peptide ID | 0.033 | 0.0015 | 0.0284 | 0.0362 | 0.003 | 0.0173 | 0.008 |
| Mass (Da) | DOWN | DOWN | UP | DOWN | DOWN | DOWN | DOWN |
| calc. | 0.59 | 0.28 | 2.28 | 0.78 | 0.42 | 0.52 | 0.62 |
| Mass (Da) | | | | | | | |
| CE-time | 39.36 | 1.92 | 145.67 | 35.95 | 37.83 | 10.38 | 13.49 |
| (min) | | | | | | | |
| Sequence | 66.53 | 6.76 | 64,00 | 46.2 | 90.8 | 20.08 | 21.69 |
| Protein name | P02452 | P02452 | P02452 | P02452 | P02452 | P02452 | P02452 |
| Start AA | 453 | 249 | 725 | 249 | 1041 | 453 | 558 |
| Stop AA | 432 | 232 | 706 | 229 | 1020 | 430 | 543 |
| Protein | Collagen alpha- | Collagen alpha- | Collagen alpha- | Collagen alpha- | Collagen alpha- | Collagen alpha- | Collagen alpha- |
| Accession | 1(I) chain | 1(I) chain | 1(I) chain | 1(I) chain | 1(I) chain | 1(I) chain | 1(I) chain |
| CAKUT | NSGEGAGSKGD | DGEAGKGRGER | IXiAKGDAGAG | NGDDGEAGKP | AEGSGRDGSGA | KGNSG EG AGS K | SGSPGPDGKTG |
| control | TGAKGEGP | GPPG (SEQ ID | AGSQGAG | GRPGERGGP | KGDRGETGP | GDTGAKGEGP | PGP (SEQ ID |
| abundance | (SEQ ID NO: | NO: 23) | (SEQ ID NO: | (SEQ ID NO: | (SEQ ID NO: | (SEQ ID NO: | NO: 28) |
| | 22) | | 24) | 25) | 26) | 27) | |
| CAKUT | 25.325039 | 21.487492 | 30.508604 | 21.92091 | 22.061558 | 22.318043 | 28.999035 |
| ease | | | | | | | |
| abundance | | | | | | | |
| Fold | 2013.887556 | 1777.834339 | 1684.728871 | 2047.9291598259 | 2085.931152 | 2199.003983 | 1435.657937 |
| change | | | | 4 | | | |
| Regulation | 2013.894531 | 1777.841431 | 1684.706909 | 2047.929565 | 2085.929932 | 2199.00293 | 1435.65918 |
| Ajusted | 20863 | 17453 | 15732 | 21342 | 21938 | 23789 | 10786 |
| p-value | | | | | | | |
| Peptide | 0.0045 | 0.002 | 0.0074 | 0.0012 | 0.0014 | 0.0025 | 0.0011 |
| ID | | | | | | | |
| Mass | DOWN | DOWN | DOWN | DOWN | DOWN | DOWN | DOWN |
| (Da) | | | | | | | |
| calc. | 0.34 | 0.26 | 0.51 | 0.22 | 0.21 | 0.26 | 0.15 |
| Mass (Da) | | | | | | | |
| CE-time | 7.91 | 8.38 | 32.81 | 0.87 | 0.52 | 2.43 | 1.33 |
| (min) | | | | | | | |
| Sequence | 22.96 | 32.4 | 64.22 | 3.97 | 2.43 | 9.45 | 8.68 |
| Protein name | P02452 | P02452 | P02452 | P02452 | P02452 | P02452 | P02452 |
| Start AA | 854 | 1039 | 843 | 810 | 1039 | 668 | 453 |
| Stop AA | 815 | 1021 | 820 | 799 | 1023 | 650 | 431 |
| Protein Accession | Collagen | Collagen | Collagen | Collagen | Collagen | Collagen | Collagen |
| | alpha- | alpha- | alpha- | alpha- | alpha- | alpha- | alpha- |
| | 1(I) chain | 1(I) chain | 1(I) chain | 1(I) chain | 1(I) chain | 1(I) chain | 1(I) chain |
| CAKUT control | GPGADGQPGAK | EGSGRDGSGAK | DGQGAKGEGD | GDRGEGPGP | SGRDGSGAKGD | GPGEAGKGEQG | GN SGEG AGSKG |
| abundance | GEGDAGAKGD | GDRGET(SEQ | AGAKGDAGPPG | (SEQ ID NO: | RGET (SEQ ID | VPGDLG (SEQ | DTGAKGEG |
| | AGPGPAGPAGP | ID NO: 30) | (SEQ ID NO: | 32) | NO: 33) | ID NO: 34) | (SEQ ID NO: |
| | GPIG (SEQ ID | | 31) | | | | 35) |
| | NO: 29) | | | | | | |
| CAKUT ease | 31.634048 | 21.453699 | 27.497255 | 27.132401 | 20.77614 | 30.881741 | 25.60898 |
| abundance | | | | | | | |
| Fold | 3416.586905 | 1860.819811 | 2149.951219 | 1179.51563 | 1674.755754 | 1749.816957 | 2086.903935 |
| change | | | | | | | |
| Regulation | 3416.559326 | 1860.830566 | 2149.956055 | 1179.521118 | 1674.767212 | 1749.778442 | 2086.921875 |
| Ajusted | 42122 | 18649 | 22992 | 5116 | 15510 | 17010 | 21956 |
| p-value | | | | | | | |
| Peptide | 0.0015 | 0.0023 | 0.0155 | 0.0014 | 0.0003 | 0.0033 | 0.0148 |
| ID | | | | | | | |
| Mass | DOWN | DOWN | DOWN | DOWN | DOWN | DOWN | UP |
| (Da) | | | | | | | |
| calc. | 0.41 | 0.43 | 0.59 | 0.42 | 0.19 | 0.43 | 2.07 |
| Mass | | | | | | | |
| (Da) | | | | | | | |
| CE-time (min) | 7.84 | 47.83 | 41.97 | 9.29 | 2.22 | 20.98 | 703.38 |
| Sequence | 19.04 | 111.32 | 70.73 | 22.18 | 11.81 | 48.99 | 340.06 |
| Protein name | P02452 | P02452 | P02452 | P02452 | P02452 | P02452 | P02452 |
| Start AA | 725 | 1041 | 810 | 451 | 249 | 455 | 249 |
| Stop AA | 705 | 1021 | 798 | 432 | 230 | 432 | 220 |
| Protein | Collagen | Collagen | Collagen | Collagen | Collagen | Collagen | Collagen |
| Accession | alpha- | alpha- | alpha- | alpha- | alpha- | alpha- | alpha- |
| | 1(1) | 1(1) | 1(1) | 10) | 10) | 1(1) | 1(1) |
| | chain | chain | chain | chain | chain | chain | chain |
| CAKUT control | NDGAKGDAGA | EGSGRDGSGAK | AGDRGEGPGP | NSGEGAGSKGD | GDDGEAGKPGR | NSGEGAGSKGD | RGPGPGKNGDD |
| abundance | GAGSQGAG | GDRGETGP | (SEQ ID NO: | TGAKGE (SEQ | GERGPGP(SEQ | TGAKGEGVG | GEAGKPGRPGE |
| | (SEQ ID NO: | (SEQ ID NO: | 38) | ID NO: 39) | ID NO: 40) | (SEQ ID NO: | RGGP(SEQ ID |
| | 36) | 37) | | | | 41) | NO: 42) |
| CAKUT | 31.345865 | 21.935715 | 27.852589 | 24.56324 | 21.382504 | 26.061628 | 20.462645 |
| ease | | | | | | | |
| abundance | | | | | | | |
| Fold | 1798.771798 | 2014.894039 | 1250.552744 | 1859.813329 | 1933.887831 | 2185.972348 | 2923.392108 |
| change | | | | | | | |
| Regulation | 1798.77124 | 2014.900146 | 1250.55896 | 1859.821167 | 1933.879761 | 2185.972168 | 2923.399658 |
| Ajusted | 17760 | 20876 | 6600 | 18627 | 19732 | 23577 | 35226 |
| p-value | | | | | | | |
| Peptide | 0.0002 | 0.003 | 0.0002 | 0.0124 | 0.003 | 0.0009 | 0.0005 |
| ID | | | | | | | |
| Mass | DOWN | UP | DOWN | DOWN | DOWN | DOWN | DOWN |
| (Da) | | | | | | | |
| calc. | 0.01 | 10.06 | 0.15 | 0.79 | 0.38 | 0.52 | 0.3 |
| Mass | | | | | | | |
| (Da) | | | | | | | |
| CE-time (min) | 0.28 | 61.55 | 1.26 | 9.37 | 1.39 | 6.25 | 5.64 |
| Sequence | 26.16 | 6.12 | 8.13 | 11.82 | 3.61 | 11.91 | 18.98 |
| Protein name | P02452 | P02452 | P02452 | P02452 | P02452 | P02452 | P02452 |
| Start AA | 539 | 1061 | 249 | 668 | 719 | 558 | 455 |
| Stop AA | 510 | 1033 | 230 | 651 | 705 | 546 | 434 |
| Protein | Collagen | Collagen | Collagen | Collagen | Collagen | Collagen | Collagen |
| Accession | alpha- | alpha- | alpha- | alpha- | alpha- | alpha- | alpha- |
| | 1(1) | 1(1) | 1(1) | 10) | 10) | 1(1) | 1(1) |
| | chain | chain | chain | chain | chain | chain | chain |
| CAKUT | ERGSG | KGDRGE | GDDGE | PGEAGK | NDGAKGDAGA | SGPDGKTGPGP | GEGAGSKGDTG |
| control | PAGPKG | TGPAG | AGKGRP | GEQGV | GAG (SEQ ID | (SEQ ID NO: | AKGEGPVG |
| abundance | SGEAGR | PGAGAP | GERGPG (SEQ | GDLG | NO: 47) | 48) | (SEQ ID NO: |
| | GEAGL | GAGPV | ID NO: 45) | (SEQ ID | | | 49) |
| | GAKG (SEQ ID | GPAG (SEQ ID | | NO: 46) | | | |
| | NO: 43) | NO: 44) | | | | | |
| CAKUT | 21.587481 | 27.791513 | 21.650738 | 31.285788 | 25.658968 | 26.928146 | 25.246342 |
| ease | | | | | | | |
| abundance | | | | | | | |
| Fold | 2761.337948 | 2496.199329 | 1949.882746 | 1708.790408 | 1285.553472 | 1194.551681 | 1968.90083951344 |
| change | | | | | | | |
| Regulation | 2761.337891 | 2496.107422 | 1949.892578 | 1708.764893 | 1285.565308 | 1194.550171 | 1968.904053 |
| Ajusted | 32876 | 28628 | 19950 | 16197 | 7437 | 5420 | 20228 |
| p-value | | | | | | | |
| Peptide | 0.0095 | 0.0014 | 0.0012 | 0.013 | 0.032 | 0.002 | 0.0041 |
| ID | | | | | | | |
| Mass | DOWN | UP | DOWN | UP | DOWN | DOWN | DOWN |
| (Da) | | | | | | | |
| calc. | 0.92 | 3.09 | 0.43 | 6.11 | 0.72 | 0.22 | 0.55 |
| Mass | | | | | | | |
| (Da) | | | | | | | |
| CE-time (min) | 24.41 | 270.26 | 3.27 | 1485.04 | 21.66 | 2.44 | 243.78 |
| Sequence | 26.47 | 87.58 | 7.64 | 243,00 | 30.07 | 11.07 | 441.98 |
| Protein name | P02452 | P02452 | P02452 | P02458 | P02458 | P02461 | P02461 |
| Start AA | 672 | 220 | 672 | 845 | 924 | 604 | 567 |
| Stop AA | 651 | 200 | 657 | 807 | 911 | 587 | 543 |
| Protein | Collagen | Collagen | Collagen | Collagen | Collagen | Collagen | Collagen |
| Accession | alpha- | alpha- | alpha- | alpha- | alpha- | alpha- | alpha- |
| | 1(1) | 1(1) | 1(1) | 1 (II) | 1 (II) | 1 (III) | 1 (HI) |
| | chain | chain | chain | chain | chain | chain | chain |
| CAKUT control | PGEAGKGEQGV | GFQGPGEGEPG | KG EQGVGDLG | GPGAGSAGARG | NGNPGPGPGPS | DGAGKNGERG | GGGSDGKPGGS |
| abundance | GDLGAGP (SEQ | ASGPMGR | AGP (SEQ ID | AGERGETGPPG | G(SEQ ID NO: | GGGGP (SEQ | QGESGRPGPG |
| | ID NO: 50) | (SEQ ID NO: | NO: 52) | PAGFAGPPGAD | 54) | ID NO: 55) | (SEQ ID NO: |
| | | 51) | | GQP (SEQ ID | | | 56) |
| | | | | NO: 53) | | | |
| CAKUT | 32.408882 | 32.201748 | 29.42359 | 32.275494 | 38.04417 | 24.07135 | 26.285912 |
| ease | | | | | | | |
| abundance | | | | | | | |
| Fold | 2046.949428 | 2025.885054 | 1522.726351 | 3423.57491177906 | 1232.542179 | 1623.723726 | 2248.994481 |
| change | | | | | | | |
| Regulation | 2046.907471 | 2025.872437 | 1522.682129 | 3423.542969 | 1232.543213 | 1623.723877 | 2248.999268 |
| Ajusted | 21320 | 21028 | 12489 | 42214 | 6196 | 14475 | 24608 |
| p-value | | | | | | | |
| Peptide | 0.0012 | 0.001 | 0.0011 | 0.001 | 0.0004 | 0.0033 | 0.0001 |
| ID | | | | | | | |
| Mass | DOWN | UP | DOWN | DOWN | DOWN | DOWN | DOWN |
| (Da) | | | | | | | |
| calc. | 0.46 | 37.08 | 0.13 | 0.21 | 0.23 | 0.54 | 0.16 |
| Mass | | | | | | | |
| (Da) | | | | | | | |
| CE-time (min) | 37.22 | 289.13 | 1.77 | 4.51 | 1.03 | 176.73 | 5.06 |
| Sequence | 81.25 | 7.8 | 13.96 | 21.95 | 4.52 | 324.49 | 32.1 |
| Protein name | P02461 | P02461 | P02461 | P02461 | P02461 | P02461 | P02461 |
| Start AA | 687 | 1054 | 687 | 936 | 806 | 477 | 840 |
| Stop AA | 664 | 1042 | 662 | 899 | 796 | 448 | 816 |
| Protein | Collagen | Collagen | Collagen | Collagen | Collagen | Collagen | Collagen |
| Accession | alpha- | alpha- | alpha- | alpha- | alpha- | alpha- | alpha- |
| | 1(111) | 1(III) | 1(III) | 1(III) | 1(111) | 1(111) | 1 (III) |
| | chain | chain | chain | chain | chain | chain | chain |
| CAKUT control | DAGAGAGGKG | AGAGHPGPGP | KG DAG AG AGG | GKDGGPAGNT | SGERGETGP | ERGEAGIGVGA | GQNGEGGKGE |
| abundance | DAGAGERGPG | (SEQ ID NO: | KG DAG AGERGP | GAPGSGVSGPK | (SEQ ID NO: | KGEDGKDGSGE | RGAGEKGEGGP |
| | (SEQ ID NO: | 58) | G (SEQ ID NO: | GDAGQPGEKGS | 61) | GANG (SEQ ID | G (SEQ ID NO: |
| | 57) | | 59) | PG (SEQ ID | | NO: 62) | 63) |
| | | | | NO: 60) | | | |
| CAKUT | 26.560427 | 26.254732 | 22.609119 | 25.760281 | 25.914463 | 24.373287 | 22.619993 |
| ease | | | | | | | |
| abundance | | | | | | | |
| Fold | 2078.925339 | 1155.530886 | 2264.041765 | 3356.550519 | 1114.489081 | 2825.269987 | 2323.042494 |
| change | | | | | | | |
| Regulation | 2078.930176 | 1155.545166 | 2264.031982 | 3356.513184 | 1114.492676 | 2825.259521 | 2323.053223 |
| Ajusted | 21830 | 4697 | 24856 | 41269 | 3917 | 33880 | 25800 |
| p-value | | | | | | | |
| Peptide | 0.0011 | 0.0009 | 0.0152 | 0.0316 | 0.0343 | 0.0011 | 0.0152 |
| ID | | | | | | | |
| Mass | UP | DOWN | DOWN | DOWN | UP | DOWN | DOWN |
| (Da) | | | | | | | |
| calc. | 23.51 | 0.22 | 0.5 | 0.52 | 3.04 | 0.08 | 0.44 |
| Mass | | | | | | | |
| (Da) | | | | | | | |
| CE-time (min) | 104.74 | 4.19 | 84.3 | 8.71 | 49.17 | 0.62 | 1.72 |
| Sequence | 4.46 | 18.94 | 167.38 | 16.81 | 16.18 | 7.88 | 3.9 |
| Protein name | P02462 | P20849 | P20908 | P27658 | Q14993 | Q07092 | Q9UMD9 |
| Start AA | 670 | 896 | 781 | 572 | 910 | 1368 | 680 |
| Stop AA | 658 | 858 | 753 | 560 | 851 | 1337 | 648 |
| Protein | Collagen | Collagen | Collagen | Collagen | Collagen | Collagen | Collagen |
| Accession | alpha- | alpha- | alpha- | alpha- | alpha- | alpha- | alpha- |
| | 1 (IV) | 1(IX) | 1(V) | l(VIII) | 1 (XIX) | 1 (XVI) | l(XVII) |
| | chain | chain | chain | chain | chain | chain | chain |
| CAKUT control | GFGPQGDRGFP | GLGDPGASYGR | GMPGADGPPGH | GPGPGPGPA | GDPGPVGEPGAM | PGGEPGTDGAA | AAGEPGPHGGV |
| abundance | G (SEQ ID NO: | NGRDGERGPGV | PGKEGGEKGGQ | (SEQ ID NO: | GLPGLEGFPGVKG | GKEGPGKQGFY | PGSVGPKGSSG |
| | 64) | AGIPGVPGPPGP | GPG (SEQ ID | 67) | DRGPAGPGIAGMS | GPGPKG (SEQ | SPGQGPG (SEQ |
| | | G(SEQ ID NO: | NO: 66) | | GKPGAGPGVGEPG | ID NO: 69) | ID NO: 70) |
| | | 65) | | | ERGPV (SEQ ID | | |
| | | | | | NO: 68) | | |
| CAKUT | 28.127964 | 27.764822 | 23.563358 | 37.29739 | 27.883848 | 23.099014 | 29.346113 |
| ease | | | | | | | |
| abundance | | | | | | | |
| Fold | 1303.60355071375 | 3648.766935 | 2679.198343 | 1157.535303 | 5508.683078 | 3021.410444 | 2890.348178 |
| change | | | | | | | |
| Regulation | 1303.584106 | 3648.751221 | 2679.198242 | 1157.533813 | 5508.611816 | 3021.369141 | 2890.253174 |
| Ajusted | 7823 | 45055 | 31488 | 4727 | 64283 | 36627 | 34805 |
| p-value | | | | | | | |
| Peptide | 0.0076 | 0.0344 | 0.0115 | 0.0233 | 0.0152 | 0.0342 | 0.0041 |
| ID | | | | | | | |
| Mass | DOWN | UP | DOWN | UP | DOWN | UP | DOWN |
| (Da) | | | | | | | |
| calc. | 0.41 | 4.09 | 0.5 | 1.07 | 0.59 | 8.72 | 0.37 |
| Mass | | | | | | | |
| (Da) | | | | | | | |
| CE-time | 33.5 | 1647.51 | 5.46 | 19.58 | 4.55 | 1433.83 | 20.11 |
| (min) | | | | | | | |
| Sequence | 81.48 | 403.12 | 10.93 | 18.23 | 7.7 | 164.51 | 54.29 |
| Protein | Q9UMD9 | P39060 | P39060 | Q96P44 | Q17RW2 | Q9BXS0 | Q8IZC6 |
| name | | | | | | | |
| Start | 998 | 1437 | 1264 | 900 | 774 | 252 | 1050 |
| AA | | | | | | | |
| Stop | 975 | 1422 | 1252 | 879 | 747 | 220 | 1022 |
| AA | | | | | | | |
| Protein | Collagen | Collagen | Collagen | Collagen | Collagen | Collagen | Collagen |
| Accession | alpha- | alpha- | alpha- | alpha- | alpha- | alpha- | alpha- |
| | 1(XVII) | 1(XVIII) | 1(XVIII) | 1(XXI) | 1(XXIV) | 1(XXV) | 1 (XXVII) |
| | chain | chain | chain | chain | chain | chain | chain |
| CAKUT | GIPSGS | SVPGPG | GMPGP | GSQGF | GKSGP | PGVPG | VPGPKG |
| control | EGGSSS | PGPGPG | GPGPG | GYGEQ | SGQTGD | EPGKGE | SGHPG |
| abundance | TMYVSG | (SEQ ID | (SEQ ID | GPGPG | PGLQGS | QGLMG | MPGGM |
| | GP | NO: | NO: | PEGP | GPGEG | PLGPPG | GTPGEP |
| | (SEQ ID | 72) | 73) | (SEQ ID | FG | QKGSIG | GPQGP |
| | NO: 71) | | | NO: | (SEQ ID | APG | (SEQ ID |
| | | | | 74) | NO: 75) | (SEQ ID | NO: 77) |
| | | | | | | NO: 76) | |
| CAKUT | 43.508652 | 39.228619 | 37.611717 | 44.165359 | 34.364258 | 30.763529 | 28.000504 |
| ease | | | | | | | |
| abundance | | | | | | | |
| Fold | 2264.974322 | 1426.672859 | 1161.512459 | 2112.902478 | 2582.1410128532 | 3082.521591 | 2696.214527 |
| change | | | | | 8 | | |
| Regulation | 2264.959473 | 1426.674927 | 1161.51001 | 2112.873535 | 2582.12793 | 3082.483398 | 2696.176025 |
| Ajusted | 24868 | 10640 | 4793 | 22377 | 29880 | 37566 | 31787 |
| p-value | | | | | | | |
| Peptide | 0.0364 | 0.0171 | 0.0171 | 0.0073 | 0.0214 | 0.0125 | 0.0012 |
| ID | | | | | | | |
| Mass | UP | DOWN | DOWN | DOWN | DOWN | DOWN | UP |
| (Da) | | | | | | | |
| calc. | 1.01 | 0.64 | 0.39 | 0.36 | 0.5 | 0.72 | 5.27 |
| Mass | | | | | | | |
| (Da) | | | | | | | |
| CE-time | 1.93 | 1.85 | 6.65 | 25.66 | 3.47 | 2.1 | 260.03 |
| (min) | | | | | | | |
| Sequence | 1.9 | 2.91 | 17.15 | 71.21 | 6.88 | 2.9 | 49.3 |
| Protein | P39060 | Q2UY09 | P08123 | P08123 | P08123 | P08123 | P08123 |
| name | | | | | | | |
| Start | 1412 | 726 | 76 | 863 | 64 | 157 | 863 |
| AA | | | | | | | |
| Stop | 1400 | 699 | 44 | 830 | 45 | 133 | 844 |
| AA | | | | | | | |
| Protein | Collagen | Collagen | Collagen | Collagen | Collagen | Collagen | Collagen |
| Accession | alpha- | alpha- | alpha- | alpha- | alpha- | alpha- | alpha- |
| | 1 (XVIII) | l(XXVIII) | 2(1) | 20) | 20) | 20) | 20) |
| | chain | chain | chain | chain | chain | chain | chain |
| CAKUT | GPGP | GPGPGY | RGPPG | RTGEVG | GGPPGR | GAGPG | GEKGS |
| control | GPGPS | GSQGI | PGRDGE | AVGPG | DGEDG | KAGED | GEAGTA |
| abundance | (SEQ ID | KGEQGQ | DGPTGP | FAGEK | TGPGP | GHGKP | GPGTGP |
| | NO: | GFPGK | PGPGPP | GPSGEA | (SEQ ID | GRGERG | (SEQ ID |
| | 78) | GT | GPGLGG | GTAGP | NO: 82) | (SEQ ID | NO: 84) |
| | | (SEQ ID | N | GTGP | | NO: 83) | |
| | | NO: 79) | (SEQ ID | (SEQ ID | | | |
| | | | NO: 80) | NO: 81) | | | |
| CAKUT | 37.374866 | 29.034859 | 29.612068 | 29.84955 | 32.759037 | 19.830038 | 31.460758 |
| ease | | | | | | | |
| abundance | | | | | | | |
| Fold | 1173.530218 | 2712.277973 | 3011.412175 | 3063.453371 | 1873.81728238453 | 2398.137397 | 1767.791137 |
| change | | | | | | | |
| Regulation | 1173.522705 | 2712.178711 | 3011.371338 | 3063.40918 | 1873.863892 | 2398.145752 | 1767.921753 |
| Ajusted | 5019 | 32038 | 36447 | 37285 | 18837 | 27115 | 17301 |
| p-value | | | | | | | |
| Peptide | 0.0364 | 0.0171 | 0.0171 | 0.0073 | 0.0214 | 0.0125 | 0.0012 |
| ID | | | | | | | |
| Mass | UP | DOWN | DOWN | DOWN | DOWN | DOWN | UP |
| (Da) | | | | | | | |
| calc. | 1.01 | 0.64 | 0.39 | 0.36 | 0.5 | 0.72 | 5.27 |
| Mass | | | | | | | |
| (Da) | | | | | | | |
| CE-time | 1.93 | 1.85 | 6.65 | 25.66 | 3.47 | 2.1 | 260.03 |
| (min) | | | | | | | |
| Sequence | 1.9 | 2.91 | 17.15 | 71.21 | 6.88 | 2.9 | 49.3 |
| Protein | P39060 | Q2UY09 | P08123 | P08123 | P08123 | P08123 | P08123 |
| name | | | | | | | |
| Start | 1412 | 726 | 76 | 863 | 64 | 157 | 863 |
| AA | | | | | | | |
| Stop | 1400 | 699 | 44 | 830 | 45 | 133 | 844 |
| AA | | | | | | | |
| Protein | Collagen | Collagen | Collagen | Collagen | Collagen | Collagen | Collagen |
| Accession | alpha- | alpha- | alpha- | alpha- | alpha- | alpha- | alpha- |
| | 1(XVIII) | 1(XXVIII) | 2(I) | 2(I} | 2(I) | 2(I) | 2(I) |
| | chain | chain | chain | chain | chain | chain | chain |
| CAKUT | GPGPG | GPGPGY | RGPPGP | RTGEVG | GGPPGR | GAGPG | GEKGSG |
| control | PGPS | GSQGI | GRDGE | AVGPG | DGEDG | KAGED | EAGTA |
| abundance | (SEQ ID | KGEQGQ | DGPTGP | FAGEKG | TGPGP | GHGKP | GPGTGP |
| | NO: 78) | GFPGK | PGPGPP | PSGEA | (SEQ ID | GRGERG | (SEQ ID |
| | | GT(SEQ ID | GPGLGG | GTAGPG | NO: 82) | (SEQ ID | NO: 84) |
| | | NO: 79) | N | TGP | | NO: 83) | |
| | | | (SEQ ID | (SEQ ID | | | |
| | | | NO: 80) | NO: 81) | | | |
| CAKUT | 37.374866 | 29.034859 | 29.612068 | 29.84955 | 32.759037 | 19.830038 | 31.460758 |
| ease | | | | | | | |
| abundance | | | | | | | |
| Fold | 1173.530218 | 2712.277973 | 3011.412175 | 3063.453371 | 1873.81728238453 | 2398.137397 | 1767.791137 |
| change | | | | | | | |
| Regulation | 1173.522705 | 2712.178711 | 3011.371338 | 3063.40918 | 1873.863892 | 2398.145752 | 1767.921753 |
| Ajusted | 5019 | 32038 | 36447 | 37285 | 18837 | 27115 | 17301 |
| p-value | | | | | | | |
| Peptide | 0.0014 | 0.0056 | 0.0316 | 0.0375 | 0.002 | 0.0059 | 0.0081 |
| ID | | | | | | | |
| Mass | DOWN | UP | DOWN | DOWN | DOWN | UP | DOWN |
| (Da) | | | | | | | |
| calc. | 0.2 | 4.44 | 0.54 | 0.68 | 0.35 | 32.04 | 0.64 |
| Mass | | | | | | | |
| (Da) | | | | | | | |
| CE-time | 3.76 | 130.65 | 34.96 | 119.22 | 4.13 | 213.38 | 1.52 |
| (min) | | | | | | | |
| Sequence | 18.42 | 29.41 | 64.35 | 176.35 | 11.68 | 6.66 | 2.37 |
| Protein | P08123 | P08123 | P29400 | A8TX70 | P02671 | P02671 | P04792 |
| name | | | | | | | |
| Start | 865 | 58 | 0 | 1514 | 253 | 270 | 202 |
| AA | | | | | | | |
| Stop | 831 | 45 | 0 | 1494 | 239 | 260 | 190 |
| AA | | | | | | | |
| Protein | Collagen | Collagen | Collagen | Collagen | Fibrinogen | Fibrinogen | Heat |
| Accession | alpha- | alpha- | alpha- | alpha- | alpha | alpha | shock |
| | 2(I) | 2(I) | 5(IV) | 5(VI) | chain | chain | protein |
| | chain | chain | chain | chain | | | beta-1 |
| CAKUT | TGEVGA | GPGPGR | QGPGP | GSGSR | SQLQKV | ELERPG | QLGGPE |
| control | VGPGF | DGEDG | PGSGPA | GAPGQY | PPEWK | GNEIT | AAKSD |
| abundance | AGEKGP | P | LEGPKG | GEKGF | ALTD | (SEQ ID | ET(SEQ ID |
| | SGEAG | (SEQ ID | NPGPQ | GDP | (SEQ ID | NO: | NO: 91) |
| | TAGPGT | NO: 86) | GPGRPG | (SEQ ID | NO: 89) | 90) | |
| | GPQG | | (SEQ ID | NO: 88) | | | |
| | (SEQ ID | | NO: 87) | | | | |
| | NO: 85) | | | | | | |
| CAKUT | 35.931652 | 27.881477 | 29.384726 | 24.300045 | 24.81127 | 27.906155 | 25.732746 |
| ease | | | | | | | |
| abundance | | | | | | | |
| Fold | 3092.432302 | 1335.56778411219 | 2968.44209927906 | 2048.918797 | 1738.92293424 | 1213.593881 | 1301.609925 |
| change | | | | | | | |
| Regulation | 3092.397705 | 1335.581543 | 2968.404053 | 2048.921143 | 1738.765259 | 1213.546631 | 1301.55603 |
| Ajusted | 37690 | 8721 | 35853 | 21353 | 16805 | 5781 | 7778 |
| p-value | | | | | | | |
| Peptide | 0.014 | 0.025 | 0.0053 | 0.0214 | 0.0012 | 0.0027 | 0.0001 |
| ID | | | | | | | |
| Mass | DOWN | UP | DOWN | UP | UP | UP | UP |
| (Da) | | | | | | | |
| calc. | 0.33 | 7.65 | 0.46 | 2.8 | 35.45 | 45.29 | 55.99 |
| Mass | | | | | | | |
| (Da) | | | | | | | |
| CE-time | 3.92 | 37.54 | 6.03 | 52.66 | 1387.42 | 1883.79 | 2172.29 |
| (min) | | | | | | | |
| Sequence | 11.93 | 4.91 | 13.13 | 18.78 | 39.14 | 41.59 | 38.8 |
| Protein | A6NCF5 | P10451 | Q9UHG2 | Q6UWH4 | P62328 | P62328 | P62328 |
| name | | | | | | | |
| Start | 448 | 290 | 238 | 67 | 44 | 44 | 44 |
| AA | | | | | | | |
| Stop | 424 | 279 | 223 | 52 | 19 | 20 | 21 |
| AA | | | | | | | |
| Protein | Kelch- | Osteopontin | ProSAAS | Protein | Thymosin | Thymosin | Thymosin |
| Accession | like | | | FAM198B | beta-4 | beta-4 | beta-4 |
| | protein | | | | | | |
| | 33 | | | | | | |
| CAKUT control abundance | GGLGET | HSHEDM | DHDVGS | VSQVGR | KKTETQ | KTETQE | TETQEK |
| | EDLLS | LVVDPK | ELPPE | ASLQH | EKNPL | KNPLPS | NPLPSK |
| | FEAYEL | (SEQ ID | GVLGA | GQAAE | PSKETI | KETIEQ | ETIEQE |
| | RTDSWT | NO: 93) | (SEQ ID | (SEQ ID | EQEKQA | EKQAG | KQAGES |
| | HL | | NO: 94) | NO: 95) | GES | ES | (SEQ ID |
| | (SEQ ID | | | | (SEQ ID | (SEQ ID | NO: 98) |
| | NO: 92) | | | | NO: 96) | NO: 97) | |
| CAKUT | 28.751198 | 20.33337 | 32.732773 | 22.598106 | 20.566191 | 21.706392 | 23.781242 |
| ease | | | | | | | |
| abundance | | | | | | | |
| Fold | 2838.334819 | 1405.666 | 1590.752566 | 1636.828131 | 2956.498925 | 2828.403962 | 2700.308999 |
| change | | | | | | | |
| Regulation | 2838.275879 | 1405.670044 | 1590.760742 | 1636.734863 | 2956.472412 | 2828.383301 | 2700.289795 |
| Ajusted | 34055 | 10250 | 13891 | 14735 | 35677 | 33930 | 31862 |
| p-value | | | | | | | |

[symbol]

indicates data missing or illegible when filed

TABLE 2
Clusters 1P

| List of peptides | Cluster | AUC |
|---|---|---|
| 31862 | 31862 | 0.95 |

TABLE 3
Clusters 2P

| List of peptides | Cluster | AUC |
|---|---|---|
| 4697, 5420, 6196, 6400, 6600, | 4697-6196 | 0.96 |
| 7437, 8721, 15510, 17010, | 5420-6196 | 0.95 |
| 17207, 17264, 19221, 20228, | 6196-19221 | 0.95 |
| 21320, 21342, 21353, 21684, | 6196-20228 | 0.98 |
| 21830, 22456, 23894, 24856, | 6196-21684 | 0.98 |
| 24868, 26070, 27115, 29894, | 6196-32876 | 0.95 |
| 31787, 32876, 33930, 34055, | 6196-33930 | 0.96 |
| 35853, 36447, 36627, 41269, | 6196-45055 | 0.95 |
| 42122, 45055 | 6196-6400 | 0.96 |
| | 6196-6600 | 0.96 |
| | 6196-7437 | 0.96 |
| | 6196-8721 | 0.98 |
| | 6400-33930 | 0.96 |
| | 7437-17264 | 0.95 |
| | 7437-21830 | 0.97 |
| | 7437-23894 | 0.96 |
| | 7437-36447 | 0.97 |
| | 8721-17010 | 0.95 |
| | 8721-17207 | 0.95 |
| | 8721-21342 | 0.95 |
| | 8721-21353 | 0.97 |
| | 8721-27115 | 0.95 |
| | 8721-31787 | 0.95 |
| | 8721-35853 | 0.96 |
| | 8721-42122 | 0.96 |
| | 15510-29894 | 0.95 |
| | 15510-33930 | 0.95 |
| | 15510-36447 | 0.95 |
| | 21320-34055 | 0.96 |
| | 21320-41269 | 0.96 |
| | 21342-21830 | 0.95 |
| | 22456-33930 | 0.96 |
| | 24856-36447 | 0.95 |
| | 24868-36447 | 0.96 |
| | 24868-45055 | 0.95 |
| | 26070-36447 | 0.95 |
| | 27115-36447 | 0.96 |
| | 36447-36627 | 0.96 |

TABLE 4
Clusters 3P

| List of peptides | Cluster | AUC |
|---|---|---|
| 2029, 4727, 5019, 5116, | 4727-25800-64283 | 0.95 |
| 5781, 7823, 10250, 10640, | 5019-17301-18649 | 0.95 |
| 11078, 14475, 15732, | 5019-17453-22992 | 0.95 |
| 16805, 17301, 17453, | 5019-18649-37566 | 0.95 |
| 18627, 18649, 18837, | 5116-18627-29880 | 0.95 |
| 20863, 20876, 21028, | 5781-25060-25800 | 0.96 |
| 21956, 22377, 22992, | 5781-25800-40022 | 0.95 |
| 23789, 24148, 24608, | 7823-25800-40022 | 0.95 |
| 25060, 25800, 29880, | 10250-25060-25800 | 0.95 |
| 31488, 32038, 33880, | 10640-25060-25800 | 0.95 |
| 34805, 35226, 35677, | 10640-25060-35677 | 0.95 |
| 36283, 37285, 37566, | 11078-16805-17453 | 0.96 |
| 40022, 64283 | 11078-17453-21028 | 0.95 |
| | 11078-17453-24148 | 0.96 |
| | 11078-17453-31488 | 0.97 |
| | 11078-17453-35677 | 0.96 |
| | 11078-17453-37285 | 0.95 |
| | 11078-18649-25800 | 0.97 |
| | 11078-18649-35677 | 0.95 |
| | 11078-25060-25800 | 0.95 |
| | 11078-25060-35677 | 0.95 |
| | 14475-16805-25060 | 0.95 |
| | 14475-17453-37566 | 0.95 |
| | 14475-20863-35677 | 0.95 |
| | 14475-22992-35677 | 0.95 |
| | 14475-23789-35677 | 0.95 |
| | 14735-17453-22992 | 0.95 |
| | 14735-25060-25800 | 0.96 |
| | 14735-25800-40022 | 0.95 |
| | 15732-25060-35677 | 0.95 |
| | 16805-17453-22992 | 0.95 |
| | 16805-20876-25800 | 0.95 |
| | 16805-25800-64283 | 0.95 |
| | 17301-23789-35677 | 0.95 |
| | 17301-25060-25800 | 0.95 |
| | 17453-22992-25060 | 0.95 |
| | 17453-22992-35677 | 0.97 |
| | 17453-24148-31488 | 0.95 |
| | 17453-24148-35677 | 0.95 |
| | 17453-25060-31488 | 0.97 |
| | 17453-25800-40022 | 0.95 |
| | 17453-31488-37566 | 0.96 |
| | 18627-23789-31488 | 0.95 |
| | 18627-25060-25800 | 0.95 |
| | 18649-25060-35677 | 0.95 |
| | 18649-25800-40022 | 0.95 |
| | 18649-31488-35677 | 0.95 |
| | 18837-23789-25800 | 0.95 |
| | 18837-25060-25800 | 0.95 |
| | 2029-5019-18649 | 0.95 |
| | 20863-25060-25800 | 0.95 |
| | 21956-25060-31488 | 0.95 |
| | 21956-35677-36283 | 0.95 |
| | 22377-25060-25800 | 0.95 |
| | 22377-25060-35677 | 0.95 |
| | 22992-35677-36283 | 0.96 |
| | 23789-25800-37566 | 0.95 |
| | 23789-25800-40022 | 0.96 |
| | 23789-32038-35677 | 0.95 |
| | 24148-25060-25800 | 0.95 |
| | 24148-25800-40022 | 0.95 |
| | 24608-25060-31488 | 0.98 |
| | 24608-25060-32038 | 0.95 |
| | 24608-29880-32038 | 0.95 |
| | 24608-31488-34805 | 0.95 |
| | 25060-25800-33880 | 0.95 |
| | 25060-25800-34805 | 0.96 |
| | 25060-25800-35226 | 0.96 |
| | 25060-25800-35677 | 0.95 |
| | 25060-25800-37285 | 0.95 |
| | 25060-25800-37566 | 0.95 |
| | 25060-25800-40022 | 0.95 |
| | 25060-25800-64283 | 0.96 |
| | 25060-31488-35677 | 0.96 |
| | 25060-35677-36283 | 0.97 |
| | 25800-35226-40022 | 0.95 |
| | 25800-40022-64283 | 0.97 |

TABLE 5
Clusters 1P + AF

| List of peptides | Cluster | AUC |
|---|---|---|
| 4727, 6400, 6600, 10786, 17760, | 4727 + AF | 0.96 |
| 21342, 21684, 31862, 45055 | 6400 + AF | 0.95 |
| | 6600 + AF | 0.95 |
| | 10786 + AF | 0.95 |
| | 17760 + AF | 0.95 |
| | 21342 + AF | 0.96 |
| | 21684 + AF | 0.96 |
| | 31862 + AF | 0.96 |
| | 45055 + AF | 0.96 |

TABLE 6
Clusters 2P + AF

| List of peptides | Cluster | AUC |
|---|---|---|
| 2029, 3917, 4697, 4793, | 2029-18627 + AF | 0.96 |
| 5019, 5116, 5420, 5781, | 2029-20228 + AF | 0.95 |
| 6196, 7437, 7823, 8721, | 2029-21320 + AF | 0.96 |
| 10250, 10640, 11078, | 2029-21830 + AF | 0.95 |
| 13891, 14475, 14735, | 2029-23894 + AF | 0.95 |
| 15510, 15732, 15884, | 2029-25800 + AF | 0.95 |
| 16197, 16805, 17010, | 2029-27115 + AF | 0.96 |
| 17207, 17264, 17301, | 2029-31488 + AF | 0.95 |
| 17453, 18627, 18649, | 2029-34055 + AF | 0.96 |
| 18837, 19221, 19732, | 2029-36283 + AF | 0.95 |
| 19950, 20228, 20643, | 2029-3917 + AF | 0.95 |
| 20863, 20876, 21028, | 2029-5019 + AF | 0.96 |
| 21076, 21320, 21353, | 2029-5116 + AF | 0.95 |
| 21830, 21938, 21956, | 2029-8721 + AF | 0.96 |
| 22377, 22456, 22992, | 3917-11078 + AF | 0.95 |
| 23577, 23789, 23894, | 3917-15732 + AF | 0.95 |
| 24148, 24421, 24608, | 3917-16805 + AF | 0.95 |
| 24856, 24868, 25060, | 3917-17301 + AF | 0.95 |
| 25170, 25301, 25800, | 3917-19221 + AF | 0.95 |
| 26070, 27115, 28628, | 3917-20228 + AF | 0.96 |
| 29880, 29894, 31488, | 3917-20863 + AF | 0.95 |
| 31787, 32038, 32876, | 3917-21028 + AF | 0.95 |
| 33930, 34055, 34805, | 3917-21353 + AF | 0.95 |
| 35226, 35677, 35853, | 3917-21830 + AF | 0.95 |
| 36283, 36447, 36627, | 3917-23789 + AF | 0.95 |
| 37285, 37566, 37690, | 3917-27115 + AF | 0.95 |
| 40022, 41269, 42122, | 3917-35677 + AF | 0.95 |
| 42214, 64283. | 3917-7823 + AF | 0.95 |
| | 4697-10250 + AF | 0.95 |
| | 4697-11078 + AF | 0.97 |
| | 4697-13891 + AF | 0.95 |
| | 4697-14475 + AF | 0.95 |
| | 4697-16805 + AF | 0.96 |
| | 4697-17010 + AF | 0.96 |
| | 4697-17207 + AF | 0.95 |
| | 4697-18627 + AF | 0.96 |
| | 4697-18649 + AF | 0.95 |
| | 4697-18837 + AF | 0.96 |
| | 4697-19221 + AF | 0.95 |
| | 4697-20228 + AF | 0.97 |
| | 4697-20643 + AF | 0.95 |
| | 4697-21320 + AF | 0.96 |
| | 4697-21830 + AF | 0.96 |
| | 4697-21956 + AF | 0.97 |
| | 4697-23789 + AF | 0.96 |
| | 4697-23894 + AF | 0.97 |
| | 4697-24856 + AF | 0.95 |
| | 4697-25800 + AF | 0.97 |
| | 4697-26070 + AF | 0.95 |
| | 4697-27115 + AF | 0.97 |
| | 4697-29880 + AF | 0.95 |
| | 4697-29894 + AF | 0.95 |
| | 4697-31488 + AF | 0.96 |
| | 4697-34055 + AF | 0.95 |
| | 4697-34805 + AF | 0.95 |
| | 4697-35677 + AF | 0.95 |
| | 4697-35853 + AF | 0.96 |
| | 4697-36283 + AF | 0.96 |
| | 4697-36627 + AF | 0.96 |
| | 4697-40022 + AF | 0.95 |
| | 4697-41269 + AF | 0.96 |
| | 4697-42122 + AF | 0.96 |
| | 4697-5019 + AF | 0.96 |
| | 4697-5116 + AF | 0.95 |
| | 4697-5781 + AF | 0.95 |
| | 4697-6196 + AF | 0.95 |
| | 4697-7823 + AF | 0.95 |
| | 4697-8721 + AF | 0.95 |
| | 4793-20228 + AF | 0.96 |
| | 4793-27115 + AF | 0.96 |
| | 4793-7437 + AF | 0.95 |
| | 4793-8721 + AF | 0.95 |
| | 5019-10250 + AF | 0.95 |
| | 5019-10640 + AF | 0.96 |
| | 5019-11078 + AF | 0.98 |
| | 5019-14475 + AF | 0.95 |
| | 5019-14735 + AF | 0.95 |
| | 5019-15510 + AF | 0.96 |
| | 5019-16805 + AF | 0.97 |
| | 5019-17207 + AF | 0.95 |
| | 5019-17264 + AF | 0.95 |
| | 5019-17301 + AF | 0.97 |
| | 5019-18627 + AF | 0.95 |
| | 5019-18649 + AF | 0.95 |
| | 5019-18837 + AF | 0.97 |
| | 5019-19221 + AF | 0.95 |
| | 5019-19950 + AF | 0.96 |
| | 5019-20228 + AF | 0.95 |
| | 5019-20643 + AF | 0.97 |
| | 5019-20876 + AF | 0.95 |
| | 5019-21028 + AF | 0.96 |
| | 5019-21076 + AF | 0.96 |
| | 5019-21320 + AF | 0.97 |
| | 5019-21956 + AF | 0.95 |
| | 5019-22456 + AF | 0.95 |
| | 5019-23789 + AF | 0.96 |
| | 5019-23894 + AF | 0.97 |
| | 5019-24421 + AF | 0.95 |
| | 5019-24856 + AF | 0.95 |
| | 5019-24868 + AF | 0.96 |
| | 5019-25060 + AF | 0.96 |
| | 5019-25170 + AF | 0.96 |
| | 5019-25301 + AF | 0.95 |
| | 5019-26070 + AF | 0.97 |
| | 5019-27115 + AF | 0.96 |
| | 5019-28628 + AF | 0.95 |
| | 5019-31488 + AF | 0.97 |
| | 5019-31787 + AF | 0.95 |
| | 5019-32038 + AF | 0.96 |
| | 5019-33930 + AF | 0.95 |
| | 5019-34055 + AF | 0.96 |
| | 5019-34805 + AF | 0.95 |
| | 5019-35226 + AF | 0.95 |
| | 5019-35677 + AF | 0.96 |
| | 5019-35853 + AF | 0.96 |
| | 5019-36283 + AF | 0.96 |
| | 5019-36447 + AF | 0.96 |
| | 5019-36627 + AF | 0.96 |
| | 5019-37566 + AF | 0.96 |
| | 5019-40022 + AF | 0.97 |
| | 5019-41269 + AF | 0.96 |
| | 5019-42122 + AF | 0.96 |
| | 5019-42214 + AF | 0.95 |
| | 5019-5781 + AF | 0.97 |
| | 5019-6196 + AF | 0.95 |
| | 5019-7437 + AF | 0.95 |
| | 5019-7823 + AF | 0.95 |
| | 5019-8721 + AF | 0.97 |
| | 5116-16805 + AF | 0.96 |
| | 5116-17264 + AF | 0.95 |
| | 5116-18627 + AF | 0.96 |
| | 5116-18837 + AF | 0.97 |
| | 5116-21320 + AF | 0.96 |
| | 5116-21956 + AF | 0.95 |
| | 5116-22456 + AF | 0.95 |
| | 5116-27115 + AF | 0.95 |
| | 5116-29880 + AF | 0.96 |
| | 5116-33930 + AF | 0.95 |
| | 5116-34805 + AF | 0.95 |
| | 5116-35677 + AF | 0.95 |
| | 5116-35853 + AF | 0.96 |
| | 5116-36627 + AF | 0.95 |
| | 5116-37566 + AF | 0.96 |
| | 5116-40022 + AF | 0.95 |
| | 5116-41269 + AF | 0.95 |
| | 5116-42122 + AF | 0.96 |
| | 5116-7437 + AF | 0.96 |
| | 5420-11078 + AF | 0.96 |
| | 5420-16805 + AF | 0.96 |
| | 5420-17010 + AF | 0.95 |
| | 5420-18627 + AF | 0.96 |
| | 5420-18649 + AF | 0.95 |
| | 5420-20228 + AF | 0.95 |
| | 5420-20643 + AF | 0.95 |
| | 5420-22377 + AF | 0.96 |
| | 5420-23894 + AF | 0.95 |
| | 5420-24856 + AF | 0.95 |
| | 5420-27115 + AF | 0.95 |
| | 5420-35853 + AF | 0.95 |
| | 5420-36627 + AF | 0.95 |
| | 5420-37566 + AF | 0.95 |
| | 5420-5781 + AF | 0.95 |
| | 5420-6196 + AF | 0.95 |
| | 5420-7437 + AF | 0.95 |
| | 5781-14475 + AF | 0.95 |
| | 5781-15732 + AF | 0.95 |
| | 5781-17010 + AF | 0.96 |
| | 5781-17264 + AF | 0.95 |
| | 5781-18627 + AF | 0.95 |
| | 5781-18837 + AF | 0.95 |
| | 5781-19221 + AF | 0.96 |
| | 5781-19950 + AF | 0.95 |
| | 5781-20228 + AF | 0.96 |
| | 5781-21320 + AF | 0.95 |
| | 5781-22456 + AF | 0.95 |
| | 5781-23577 + AF | 0.95 |
| | 5781-24856 + AF | 0.95 |
| | 5781-25060 + AF | 0.96 |
| | 5781-25800 + AF | 0.96 |
| | 5781-27115 + AF | 0.96 |
| | 5781-31488 + AF | 0.96 |
| | 5781-34055 + AF | 0.95 |
| | 5781-35853 + AF | 0.95 |
| | 5781-36283 + AF | 0.96 |
| | 5781-36627 + AF | 0.96 |
| | 5781-42122 + AF | 0.95 |
| | 5781-7437 + AF | 0.95 |
| | 5781-8721 + AF | 0.97 |
| | 6196-11078 + AF | 0.95 |
| | 6196-20228 + AF | 0.97 |
| | 6196-21320 + AF | 0.96 |
| | 6196-27115 + AF | 0.96 |
| | 6196-31488 + AF | 0.95 |
| | 6196-33930 + AF | 0.95 |
| | 6196-35853 + AF | 0.95 |
| | 6196-42122 + AF | 0.95 |
| | 6196-7437 + AF | 0.95 |
| | 6196-8721 + AF | 0.97 |
| | 7437-11078 + AF | 0.97 |
| | 7437-13891 + AF | 0.95 |
| | 7437-14475 + AF | 0.96 |
| | 7437-15510 + AF | 0.95 |
| | 7437-15884 + AF | 0.95 |
| | 7437-16805 + AF | 0.97 |
| | 7437-17010 + AF | 0.96 |
| | 7437-17207 + AF | 0.96 |
| | 7437-17264 + AF | 0.96 |
| | 7437-17301 + AF | 0.96 |
| | 7437-18627 + AF | 0.95 |
| | 7437-18649 + AF | 0.96 |
| | 7437-18837 + AF | 0.96 |
| | 7437-19221 + AF | 0.95 |
| | 7437-20228 + AF | 0.97 |
| | 7437-20643 + AF | 0.95 |
| | 7437-20863 + AF | 0.95 |
| | 7437-20876 + AF | 0.96 |
| | 7437-21320 + AF | 0.96 |
| | 7437-21830 + AF | 0.97 |
| | 7437-21938 + AF | 0.95 |
| | 7437-22456 + AF | 0.95 |
| | 7437-23789 + AF | 0.96 |
| | 7437-23894 + AF | 0.97 |
| | 7437-24148 + AF | 0.96 |
| | 7437-24421 + AF | 0.95 |
| | 7437-24608 + AF | 0.95 |
| | 7437-24856 + AF | 0.95 |
| | 7437-24868 + AF | 0.95 |
| | 7437-25060 + AF | 0.96 |
| | 7437-25170 + AF | 0.96 |
| | 7437-25301 + AF | 0.95 |
| | 7437-27115 + AF | 0.96 |
| | 7437-29894 + AF | 0.96 |
| | 7437-31488 + AF | 0.95 |
| | 7437-31787 + AF | 0.95 |
| | 7437-34055 + AF | 0.95 |
| | 7437-35226 + AF | 0.95 |
| | 7437-35677 + AF | 0.95 |
| | 7437-35853 + AF | 0.96 |
| | 7437-36283 + AF | 0.95 |
| | 7437-36447 + AF | 0.96 |
| | 7437-36627 + AF | 0.96 |
| | 7437-37566 + AF | 0.95 |
| | 7437-40022 + AF | 0.96 |
| | 7437-41269 + AF | 0.96 |
| | 7437-42122 + AF | 0.96 |
| | 7437-42214 + AF | 0.95 |
| | 7437-8721 + AF | 0.95 |
| | 7823-11078 + AF | 0.95 |
| | 7823-14475 + AF | 0.95 |
| | 7823-16197 + AF | 0.96 |
| | 7823-17010 + AF | 0.95 |
| | 7823-17301 + AF | 0.95 |
| | 7823-18627 + AF | 0.95 |
| | 7823-18837 + AF | 0.95 |
| | 7823-20228 + AF | 0.95 |
| | 7823-21320 + AF | 0.96 |
| | 7823-23894 + AF | 0.95 |
| | 7823-25800 + AF | 0.96 |
| | 7823-27115 + AF | 0.95 |
| | 7823-31488 + AF | 0.95 |
| | 7823-32038 + AF | 0.95 |
| | 7823-34055 + AF | 0.98 |
| | 7823-36627 + AF | 0.95 |
| | 7823-8721 + AF | 0.95 |
| | 8721-10250 + AF | 0.96 |
| | 8721-11078 + AF | 0.98 |
| | 8721-13891 + AF | 0.97 |
| | 8721-14475 + AF | 0.95 |
| | 8721-15510 + AF | 0.95 |
| | 8721-15732 + AF | 0.97 |
| | 8721-15884 + AF | 0.96 |
| | 8721-16197 + AF | 0.95 |
| | 8721-16805 + AF | 0.98 |
| | 8721-17010 + AF | 0.97 |
| | 8721-17207 + AF | 0.95 |
| | 8721-17264 + AF | 0.96 |
| | 8721-17301 + AF | 0.96 |
| | 8721-18627 + AF | 0.97 |
| | 8721-18649 + AF | 0.95 |
| | 8721-18837 + AF | 0.96 |
| | 8721-19221 + AF | 0.96 |
| | 8721-19950 + AF | 0.95 |
| | 8721-20228 + AF | 0.97 |
| | 8721-20643 + AF | 0.96 |
| | 8721-20876 + AF | 0.97 |
| | 8721-21076 + AF | 0.95 |
| | 8721-21320 + AF | 0.97 |
| | 8721-21830 + AF | 0.96 |
| | 8721-21956 + AF | 0.95 |
| | 8721-22377 + AF | 0.95 |
| | 8721-22456 + AF | 0.95 |
| | 8721-22992 + AF | 0.95 |
| | 8721-23577 + AF | 0.95 |
| | 8721-23789 + AF | 0.97 |
| | 8721-23894 + AF | 0.96 |
| | 8721-24148 + AF | 0.96 |
| | 8721-24421 + AF | 0.97 |
| | 8721-24856 + AF | 0.97 |
| | 8721-25060 + AF | 0.96 |
| | 8721-25170 + AF | 0.97 |
| | 8721-25301 + AF | 0.96 |
| | 8721-26070 + AF | 0.96 |
| | 8721-27115 + AF | 0.97 |
| | 8721-29880 + AF | 0.97 |
| | 8721-31787 + AF | 0.95 |
| | 8721-32038 + AF | 0.95 |
| | 8721-34055 + AF | 0.95 |
| | 8721-34805 + AF | 0.97 |
| | 8721-35677 + AF | 0.95 |
| | 8721-35853 + AF | 0.95 |
| | 8721-36283 + AF | 0.96 |
| | 8721-36447 + AF | 0.95 |
| | 8721-36627 + AF | 0.95 |
| | 8721-37285 + AF | 0.96 |
| | 8721-37566 + AF | 0.96 |
| | 8721-40022 + AF | 0.96 |
| | 8721-41269 + AF | 0.96 |
| | 8721-42122 + AF | 0.97 |
| | 8721-42214 + AF | 0.96 |
| | 10250-11078 + AF | 0.95 |
| | 10250-16805 + AF | 0.95 |
| | 10250-17010 + AF | 0.95 |
| | 10250-18627 + AF | 0.96 |
| | 10250-19950 + AF | 0.95 |
| | 10250-21320 + AF | 0.97 |
| | 10250-21956 + AF | 0.96 |
| | 10250-25060 + AF | 0.95 |
| | 10250-27115 + AF | 0.95 |
| | 10250-34055 + AF | 0.96 |
| | 10250-36447 + AF | 0.95 |
| | 10250-40022 + AF | 0.95 |
| | 10640-18837 + AF | 0.95 |
| | 10640-20228 + AF | 0.95 |
| | 10640-21320 + AF | 0.95 |
| | 10640-25800 + AF | 0.95 |
| | 11078-13891 + AF | 0.95 |
| | 11078-14475 + AF | 0.96 |
| | 11078-14735 + AF | 0.95 |
| | 11078-15510 + AF | 0.96 |
| | 11078-15732 + AF | 0.95 |
| | 11078-16805 + AF | 0.96 |
| | 11078-17010 + AF | 0.97 |
| | 11078-17264 + AF | 0.97 |
| | 11078-17301 + AF | 0.96 |
| | 11078-17453 + AF | 0.96 |
| | 11078-18649 + AF | 0.95 |
| | 11078-18837 + AF | 0.98 |
| | 11078-19221 + AF | 0.96 |
| | 11078-19950 + AF | 0.96 |
| | 11078-20228 + AF | 0.97 |
| | 11078-20643 + AF | 0.97 |
| | 11078-20876 + AF | 0.96 |
| | 11078-21028 + AF | 0.96 |
| | 11078-21076 + AF | 0.96 |
| | 11078-21320 + AF | 0.97 |
| | 11078-21353 + AF | 0.95 |
| | 11078-21830 + AF | 0.97 |
| | 11078-21956 + AF | 0.96 |
| | 11078-22377 + AF | 0.95 |
| | 11078-22456 + AF | 0.95 |
| | 11078-22992 + AF | 0.95 |
| | 11078-23577 + AF | 0.95 |
| | 11078-23789 + AF | 0.96 |
| | 11078-23894 + AF | 0.97 |
| | 11078-24148 + AF | 0.96 |
| | 11078-24421 + AF | 0.96 |
| | 11078-24608 + AF | 0.95 |
| | 11078-24856 + AF | 0.97 |
| | 11078-24868 + AF | 0.96 |
| | 11078-25060 + AF | 0.97 |
| | 11078-25170 + AF | 0.96 |
| | 11078-25301 + AF | 0.96 |
| | 11078-25800 + AF | 0.97 |
| | 11078-26070 + AF | 0.96 |
| | 11078-27115 + AF | 0.97 |
| | 11078-29880 + AF | 0.97 |
| | 11078-29894 + AF | 0.97 |
| | 11078-31488 + AF | 0.96 |
| | 11078-31787 + AF | 0.95 |
| | 11078-32038 + AF | 0.95 |
| | 11078-32876 + AF | 0.96 |
| | 11078-33930 + AF | 0.96 |
| | 11078-34055 + AF | 0.97 |
| | 11078-34805 + AF | 0.97 |
| | 11078-35677 + AF | 0.97 |
| | 11078-36283 + AF | 0.95 |
| | 11078-36447 + AF | 0.96 |
| | 11078-36627 + AF | 0.97 |
| | 11078-37285 + AF | 0.96 |
| | 11078-37566 + AF | 0.95 |
| | 11078-37690 + AF | 0.95 |
| | 11078-40022 + AF | 0.96 |
| | 11078-41269 + AF | 0.96 |
| | 11078-42122 + AF | 0.98 |
| | 11078-42214 + AF | 0.96 |
| | 13891-17301 + AF | 0.95 |
| | 13891-21320 + AF | 0.97 |
| | 14475-16805 + AF | 0.95 |
| | 14475-17264 + AF | 0.96 |
| | 14475-17301 + AF | 0.95 |
| | 14475-18837 + AF | 0.95 |
| | 14475-19221 + AF | 0.95 |
| | 14475-20228 + AF | 0.96 |
| | 14475-20643 + AF | 0.95 |
| | 14475-20863 + AF | 0.95 |
| | 14475-21076 + AF | 0.95 |
| | 14475-21320 + AF | 0.96 |
| | 14475-23789 + AF | 0.95 |
| | 14475-23894 + AF | 0.96 |
| | 14475-24421 + AF | 0.95 |
| | 14475-24856 + AF | 0.95 |
| | 14475-25060 + AF | 0.95 |
| | 14475-25170 + AF | 0.95 |
| | 14475-27115 + AF | 0.97 |
| | 14475-29894 + AF | 0.95 |
| | 14475-33930 + AF | 0.95 |
| | 14475-34055 + AF | 0.96 |
| | 14475-34805 + AF | 0.95 |
| | 14475-35677 + AF | 0.95 |
| | 14475-35853 + AF | 0.95 |
| | 14475-36283 + AF | 0.95 |
| | 14475-36627 + AF | 0.96 |
| | 14475-37566 + AF | 0.96 |
| | 14475-40022 + AF | 0.95 |
| | 14475-41269 + AF | 0.95 |
| | 14475-42214 + AF | 0.95 |
| | 14735-17264 + AF | 0.95 |
| | 14735-20876 + AF | 0.95 |
| | 14735-21320 + AF | 0.95 |
| | 14735-27115 + AF | 0.95 |
| | 14735-32038 + AF | 0.95 |
| | 14735-36447 + AF | 0.96 |
| | 14735-42122 + AF | 0.95 |
| | 15510-27115 + AF | 0.95 |
| | 15510-41269 + AF | 0.95 |
| | 15510-42122 + AF | 0.95 |
| | 15732-17010 + AF | 0.95 |
| | 15732-20228 + AF | 0.96 |
| | 15732-21320 + AF | 0.95 |
| | 15732-21830 + AF | 0.95 |
| | 15732-23894 + AF | 0.95 |
| | 15732-25060 + AF | 0.95 |
| | 15732-25170 + AF | 0.95 |
| | 15732-27115 + AF | 0.95 |
| | 15732-35677 + AF | 0.95 |
| | 15732-36283 + AF | 0.95 |
| | 15732-36627 + AF | 0.96 |
| | 16197-17301 + AF | 0.95 |
| | 16197-21320 + AF | 0.96 |
| | 16805-17010 + AF | 0.96 |
| | 16805-17264 + AF | 0.95 |
| | 16805-19221 + AF | 0.98 |
| | 16805-20228 + AF | 0.97 |
| | 16805-20863 + AF | 0.95 |
| | 16805-21320 + AF | 0.96 |
| | 16805-21353 + AF | 0.95 |
| | 16805-21956 + AF | 0.95 |
| | 16805-22456 + AF | 0.95 |
| | 16805-24148 + AF | 0.95 |
| | 16805-24421 + AF | 0.96 |
| | 16805-25060 + AF | 0.95 |
| | 16805-25800 + AF | 0.97 |
| | 16805-27115 + AF | 0.97 |
| | 16805-29880 + AF | 0.96 |
| | 16805-29894 + AF | 0.95 |
| | 16805-31488 + AF | 0.95 |
| | 16805-32038 + AF | 0.97 |
| | 16805-34805 + AF | 0.95 |
| | 16805-36627 + AF | 0.96 |
| | 16805-41269 + AF | 0.95 |
| | 16805-42122 + AF | 0.95 |
| | 17010-17301 + AF | 0.95 |
| | 17010-18837 + AF | 0.97 |
| | 17010-20228 + AF | 0.96 |
| | 17010-20863 + AF | 0.96 |
| | 17010-21830 + AF | 0.95 |
| | 17010-22456 + AF | 0.95 |
| | 17010-23789 + AF | 0.95 |
| | 17010-24856 + AF | 0.95 |
| | 17010-27115 + AF | 0.95 |
| | 17010-35853 + AF | 0.95 |
| | 17010-36627 + AF | 0.95 |
| | 17010-37566 + AF | 0.95 |
| | 17010-42214 + AF | 0.95 |
| | 17207-17301 + AF | 0.96 |
| | 17207-18837 + AF | 0.96 |
| | 17207-21320 + AF | 0.97 |
| | 17207-23577 + AF | 0.95 |
| | 17207-25301 + AF | 0.95 |
| | 17207-27115 + AF | 0.95 |
| | 17207-29880 + AF | 0.96 |
| | 17207-34055 + AF | 0.96 |
| | 17207-35853 + AF | 0.95 |
| | 17207-36627 + AF | 0.96 |
| | 17207-37566 + AF | 0.95 |
| | 17207-41269 + AF | 0.95 |
| | 17207-42122 + AF | 0.96 |
| | 17264-18837 + AF | 0.96 |
| | 17264-19221 + AF | 0.95 |
| | 17264-20228 + AF | 0.95 |
| | 17264-20863 + AF | 0.95 |
| | 17264-21320 + AF | 0.96 |
| | 17264-21956 + AF | 0.95 |
| | 17264-23577 + AF | 0.95 |
| | 17264-23789 + AF | 0.95 |
| | 17264-24421 + AF | 0.95 |
| | 17264-25060 + AF | 0.95 |
| | 17264-25800 + AF | 0.95 |
| | 17264-26070 + AF | 0.95 |
| | 17264-27115 + AF | 0.96 |
| | 17264-31488 + AF | 0.95 |
| | 17264-32038 + AF | 0.96 |
| | 17264-34055 + AF | 0.95 |
| | 17264-34805 + AF | 0.96 |
| | 17264-35853 + AF | 0.95 |
| | 17264-36283 + AF | 0.95 |
| | 17264-36627 + AF | 0.96 |
| | 17264-40022 + AF | 0.95 |
| | 17301-18649 + AF | 0.95 |
| | 17301-18837 + AF | 0.96 |
| | 17301-20228 + AF | 0.96 |
| | 17301-20876 + AF | 0.95 |
| | 17301-21320 + AF | 0.97 |
| | 17301-21830 + AF | 0.96 |
| | 17301-21956 + AF | 0.95 |
| | 17301-22377 + AF | 0.96 |
| | 17301-23577 + AF | 0.95 |
| | 17301-23789 + AF | 0.95 |
| | 17301-23894 + AF | 0.96 |
| | 17301-24421 + AF | 0.95 |
| | 17301-24856 + AF | 0.95 |
| | 17301-25060 + AF | 0.95 |
| | 17301-25800 + AF | 0.97 |
| | 17301-26070 + AF | 0.96 |
| | 17301-27115 + AF | 0.96 |
| | 17301-34805 + AF | 0.95 |
| | 17301-35853 + AF | 0.95 |
| | 17301-36627 + AF | 0.96 |
| | 17301-40022 + AF | 0.95 |
| | 17301-42214 + AF | 0.95 |
| | 17453-31488 + AF | 0.95 |
| | 18627-19221 + AF | 0.96 |
| | 18627-19950 + AF | 0.96 |
| | 18627-20228 + AF | 0.96 |
| | 18627-21076 + AF | 0.95 |
| | 18627-21320 + AF | 0.96 |
| | 18627-21956 + AF | 0.95 |
| | 18627-25060 + AF | 0.95 |
| | 18627-25800 + AF | 0.95 |
| | 18627-27115 + AF | 0.95 |
| | 18627-29880 + AF | 0.95 |
| | 18627-29894 + AF | 0.95 |
| | 18627-32038 + AF | 0.96 |
| | 18627-33930 + AF | 0.95 |
| | 18627-34055 + AF | 0.97 |
| | 18627-34805 + AF | 0.95 |
| | 18627-35226 + AF | 0.96 |
| | 18627-35677 + AF | 0.95 |
| | 18627-35853 + AF | 0.97 |
| | 18627-36627 + AF | 0.96 |
| | 18627-41269 + AF | 0.95 |
| | 18627-42122 + AF | 0.95 |
| | 18649-21320 + AF | 0.95 |
| | 18649-29894 + AF | 0.95 |
| | 18837-19950 + AF | 0.95 |
| | 18837-20876 + AF | 0.95 |
| | 18837-21076 + AF | 0.95 |
| | 18837-21320 + AF | 0.95 |
| | 18837-22456 + AF | 0.95 |
| | 18837-23577 + AF | 0.95 |
| | 18837-25060 + AF | 0.96 |
| | 18837-25301 + AF | 0.95 |
| | 18837-25800 + AF | 0.96 |
| | 18837-27115 + AF | 0.96 |
| | 18837-31488 + AF | 0.95 |
| | 18837-32038 + AF | 0.95 |
| | 18837-33930 + AF | 0.95 |
| | 18837-34055 + AF | 0.95 |
| | 18837-34805 + AF | 0.96 |
| | 18837-35853 + AF | 0.96 |
| | 18837-36283 + AF | 0.97 |
| | 18837-36627 + AF | 0.96 |
| | 18837-37285 + AF | 0.96 |
| | 18837-37690 + AF | 0.95 |
| | 18837-41269 + AF | 0.97 |
| | 19221-21320 + AF | 0.97 |
| | 19221-21956 + AF | 0.95 |
| | 19221-22456 + AF | 0.96 |
| | 19221-23894 + AF | 0.95 |
| | 19221-24148 + AF | 0.96 |
| | 19221-24421 + AF | 0.96 |
| | 19221-24856 + AF | 0.95 |
| | 19221-24868 + AF | 0.95 |
| | 19221-27115 + AF | 0.95 |
| | 19221-31488 + AF | 0.95 |
| | 19221-31787 + AF | 0.96 |
| | 19221-35853 + AF | 0.95 |
| | 19221-36283 + AF | 0.96 |
| | 19221-36627 + AF | 0.95 |
| | 19221-37285 + AF | 0.96 |
| | 19221-37566 + AF | 0.95 |
| | 19221-41269 + AF | 0.95 |
| | 19732-20228 + AF | 0.95 |
| | 19950-20228 + AF | 0.95 |
| | 19950-21320 + AF | 0.95 |
| | 19950-24856 + AF | 0.95 |
| | 19950-25800 + AF | 0.96 |
| | 19950-27115 + AF | 0.95 |
| | 19950-34055 + AF | 0.95 |
| | 19950-35853 + AF | 0.95 |
| | 19950-36627 + AF | 0.95 |
| | 20228-20876 + AF | 0.96 |
| | 20228-21320 + AF | 0.97 |
| | 20228-21830 + AF | 0.95 |
| | 20228-22456 + AF | 0.97 |
| | 20228-22992 + AF | 0.95 |
| | 20228-23577 + AF | 0.97 |
| | 20228-23789 + AF | 0.96 |
| | 20228-23894 + AF | 0.95 |
| | 20228-24856 + AF | 0.96 |
| | 20228-25060 + AF | 0.97 |
| | 20228-25301 + AF | 0.95 |
| | 20228-25800 + AF | 0.95 |
| | 20228-27115 + AF | 0.97 |
| | 20228-31488 + AF | 0.96 |
| | 20228-32876 + AF | 0.95 |
| | 20228-34055 + AF | 0.95 |
| | 20228-34805 + AF | 0.95 |
| | 20228-35677 + AF | 0.95 |
| | 20228-35853 + AF | 0.97 |
| | 20228-36283 + AF | 0.98 |
| | 20228-36447 + AF | 0.95 |
| | 20228-36627 + AF | 0.96 |
| | 20228-37285 + AF | 0.96 |
| | 20228-37566 + AF | 0.95 |
| | 20228-41269 + AF | 0.97 |
| | 20228-42214 + AF | 0.95 |
| | 20643-21320 + AF | 0.96 |
| | 20643-23577 + AF | 0.95 |
| | 20643-27115 + AF | 0.95 |
| | 20643-35853 + AF | 0.95 |
| | 20643-36283 + AF | 0.96 |
| | 20643-36447 + AF | 0.96 |
| | 20643-41269 + AF | 0.96 |
| | 20863-20876 + AF | 0.95 |
| | 20863-21320 + AF | 0.97 |
| | 20863-25060 + AF | 0.95 |
| | 20863-25170 + AF | 0.96 |
| | 20863-25301 + AF | 0.95 |
| | 20863-27115 + AF | 0.96 |
| | 20863-34055 + AF | 0.95 |
| | 20863-35853 + AF | 0.95 |
| | 20863-36627 + AF | 0.95 |
| | 20863-37285 + AF | 0.95 |
| | 20863-42122 + AF | 0.95 |
| | 20876-21320 + AF | 0.97 |
| | 20876-21956 + AF | 0.95 |
| | 20876-27115 + AF | 0.96 |
| | 20876-31488 + AF | 0.95 |
| | 20876-34805 + AF | 0.95 |
| | 20876-36627 + AF | 0.95 |
| | 20876-42122 + AF | 0.95 |
| | 21028-31488 + AF | 0.95 |
| | 21076-21320 + AF | 0.97 |
| | 21076-25060 + AF | 0.95 |
| | 21076-27115 + AF | 0.97 |
| | 21076-35853 + AF | 0.96 |
| | 21076-36283 + AF | 0.95 |
| | 21076-36447 + AF | 0.96 |
| | 21076-41269 + AF | 0.95 |
| | 21076-42122 + AF | 0.96 |
| | 21320-21830 + AF | 0.97 |
| | 21320-21956 + AF | 0.96 |
| | 21320-22377 + AF | 0.95 |
| | 21320-22456 + AF | 0.96 |
| | 21320-22992 + AF | 0.95 |
| | 21320-23577 + AF | 0.95 |
| | 21320-23789 + AF | 0.97 |
| | 21320-23894 + AF | 0.97 |
| | 21320-24148 + AF | 0.96 |
| | 21320-24421 + AF | 0.97 |
| | 21320-24856 + AF | 0.97 |
| | 21320-24868 + AF | 0.96 |
| | 21320-25060 + AF | 0.97 |
| | 21320-25170 + AF | 0.95 |
| | 21320-25301 + AF | 0.97 |
| | 21320-25800 + AF | 0.97 |
| | 21320-26070 + AF | 0.97 |
| | 21320-27115 + AF | 0.97 |
| | 21320-28628 + AF | 0.95 |
| | 21320-29880 + AF | 0.97 |
| | 21320-31488 + AF | 0.98 |
| | 21320-32038 + AF | 0.96 |
| | 21320-33930 + AF | 0.96 |
| | 21320-34055 + AF | 0.97 |
| | 21320-34805 + AF | 0.97 |
| | 21320-35677 + AF | 0.95 |
| | 21320-35853 + AF | 0.97 |
| | 21320-36283 + AF | 0.97 |
| | 21320-36447 + AF | 0.96 |
| | 21320-36627 + AF | 0.97 |
| | 21320-37285 + AF | 0.96 |
| | 21320-37566 + AF | 0.95 |
| | 21320-40022 + AF | 0.96 |
| | 21320-41269 + AF | 0.97 |
| | 21320-42122 + AF | 0.97 |
| | 21320-42214 + AF | 0.95 |
| | 21830-22456 + AF | 0.96 |
| | 21830-24421 + AF | 0.95 |
| | 21830-32038 + AF | 0.95 |
| | 21830-34055 + AF | 0.95 |
| | 21830-36627 + AF | 0.95 |
| | 21830-37566 + AF | 0.95 |
| | 21830-41269 + AF | 0.95 |
| | 21956-22456 + AF | 0.95 |
| | 21956-23894 + AF | 0.96 |
| | 21956-25060 + AF | 0.95 |
| | 21956-26070 + AF | 0.95 |
| | 21956-27115 + AF | 0.95 |
| | 21956-28628 + AF | 0.95 |
| | 21956-29880 + AF | 0.96 |
| | 21956-29894 + AF | 0.96 |
| | 21956-31488 + AF | 0.97 |
| | 21956-32038 + AF | 0.95 |
| | 21956-34055 + AF | 0.97 |
| | 21956-35853 + AF | 0.96 |
| | 21956-36283 + AF | 0.95 |
| | 21956-37285 + AF | 0.96 |
| | 21956-42122 + AF | 0.95 |
| | 22377-42122 + AF | 0.95 |
| | 22456-24856 + AF | 0.95 |
| | 22456-25170 + AF | 0.95 |
| | 22456-27115 + AF | 0.95 |
| | 22456-29880 + AF | 0.95 |
| | 22456-31488 + AF | 0.95 |
| | 22456-33930 + AF | 0.95 |
| | 22456-34805 + AF | 0.95 |
| | 22456-42122 + AF | 0.96 |
| | 22456-42214 + AF | 0.95 |
| | 23577-27115 + AF | 0.95 |
| | 23577-33930 + AF | 0.95 |
| | 23577-35677 + AF | 0.95 |
| | 23577-35853 + AF | 0.95 |
| | 23577-42122 + AF | 0.95 |
| | 23789-23894 + AF | 0.95 |
| | 23789-25800 + AF | 0.95 |
| | 23789-27115 + AF | 0.95 |
| | 23789-29880 + AF | 0.95 |
| | 23789-31488 + AF | 0.95 |
| | 23789-31787 + AF | 0.95 |
| | 23789-35853 + AF | 0.95 |
| | 23789-36627 + AF | 0.95 |
| | 23789-37566 + AF | 0.96 |
| | 23789-42122 + AF | 0.96 |
| | 23894-27115 + AF | 0.95 |
| | 23894-29880 + AF | 0.95 |
| | 23894-31488 + AF | 0.95 |
| | 23894-32038 + AF | 0.96 |
| | 23894-33930 + AF | 0.95 |
| | 23894-34805 + AF | 0.95 |
| | 23894-36283 + AF | 0.95 |
| | 23894-40022 + AF | 0.95 |
| | 23894-42122 + AF | 0.95 |
| | 24148-27115 + AF | 0.95 |
| | 24148-32038 + AF | 0.96 |
| | 24148-36627 + AF | 0.95 |
| | 24148-42122 + AF | 0.95 |
| | 24421-27115 + AF | 0.95 |
| | 24421-35853 + AF | 0.95 |
| | 24421-37566 + AF | 0.95 |
| | 24421-41269 + AF | 0.96 |
| | 24856-25060 + AF | 0.95 |
| | 24856-32038 + AF | 0.95 |
| | 24856-34055 + AF | 0.95 |
| | 24856-34805 + AF | 0.95 |
| | 24856-36283 + AF | 0.95 |
| | 24856-41269 + AF | 0.95 |
| | 24868-27115 + AF | 0.95 |
| | 25060-27115 + AF | 0.97 |
| | 25060-35853 + AF | 0.95 |
| | 25060-36283 + AF | 0.95 |
| | 25060-36627 + AF | 0.95 |
| | 25060-37566 + AF | 0.95 |
| | 25060-42214 + AF | 0.95 |
| | 25170-27115 + AF | 0.96 |
| | 25170-31488 + AF | 0.95 |
| | 25170-34805 + AF | 0.95 |
| | 25170-35853 + AF | 0.95 |
| | 25170-36283 + AF | 0.95 |
| | 25170-36627 + AF | 0.96 |
| | 25800-27115 + AF | 0.95 |
| | 25800-29880 + AF | 0.95 |
| | 25800-32876 + AF | 0.95 |
| | 25800-34805 + AF | 0.95 |
| | 25800-35677 + AF | 0.96 |
| | 25800-35853 + AF | 0.96 |
| | 25800-37566 + AF | 0.96 |
| | 25800-40022 + AF | 0.95 |
| | 25800-42122 + AF | 0.96 |
| | 25800-42214 + AF | 0.96 |
| | 26070-27115 + AF | 0.95 |
| | 26070-29880 + AF | 0.95 |
| | 26070-31488 + AF | 0.95 |
| | 26070-34805 + AF | 0.96 |
| | 26070-35853 + AF | 0.95 |
| | 27115-29880 + AF | 0.95 |
| | 27115-31488 + AF | 0.96 |
| | 27115-32038 + AF | 0.95 |
| | 27115-33930 + AF | 0.95 |
| | 27115-34055 + AF | 0.95 |
| | 27115-34805 + AF | 0.96 |
| | 27115-35677 + AF | 0.96 |
| | 27115-35853 + AF | 0.95 |
| | 27115-36283 + AF | 0.96 |
| | 27115-36447 + AF | 0.96 |
| | 27115-36627 + AF | 0.95 |
| | 27115-37566 + AF | 0.96 |
| | 27115-37690 + AF | 0.95 |
| | 27115-40022 + AF | 0.97 |
| | 27115-41269 + AF | 0.96 |
| | 27115-42122 + AF | 0.96 |
| | 27115-42214 + AF | 0.95 |
| | 29880-33930 + AF | 0.95 |
| | 29880-34055 + AF | 0.95 |
| | 29880-41269 + AF | 0.95 |
| | 29880-42122 + AF | 0.95 |
| | 29894-41269 + AF | 0.95 |
| | 29894-42122 + AF | 0.95 |
| | 31488-33930 + AF | 0.95 |
| | 31488-34805 + AF | 0.96 |
| | 31488-35853 + AF | 0.96 |
| | 31488-37566 + AF | 0.95 |
| | 31488-42214 + AF | 0.96 |
| | 32038-35677 + AF | 0.95 |
| | 32038-36447 + AF | 0.95 |
| | 32038-40022 + AF | 0.97 |
| | 32038-41269 + AF | 0.95 |
| | 32038-42122 + AF | 0.96 |
| | 33930-34055 + AF | 0.97 |
| | 33930-36283 + AF | 0.95 |
| | 33930-41269 + AF | 0.95 |
| | 34055-35677 + AF | 0.97 |
| | 34055-42214 + AF | 0.95 |
| | 34055-64283 + AF | 0.96 |
| | 34805-35677 + AF | 0.95 |
| | 34805-35853 + AF | 0.95 |
| | 34805-36447 + AF | 0.96 |
| | 34805-36627 + AF | 0.96 |
| | 34805-40022 + AF | 0.95 |
| | 34805-41269 + AF | 0.96 |
| | 34805-42122 + AF | 0.96 |
| | 35677-36283 + AF | 0.96 |
| | 35853-40022 + AF | 0.96 |
| | 35853-41269 + AF | 0.95 |
| | 35853-42122 + AF | 0.96 |
| | 36283-36627 + AF | 0.95 |
| | 36283-37566 + AF | 0.97 |
| | 36283-40022 + AF | 0.95 |
| | 36283-41269 + AF | 0.96 |
| | 36283-42122 + AF | 0.95 |
| | 36283-42214 + AF | 0.96 |
| | 36627-40022 + AF | 0.96 |
| | 36627-41269 + AF | 0.96 |
| | 36627-42122 + AF | 0.96 |
| | 37285-42122 + AF | 0.96 |

TABLE 7
Thymosin-β4

| Protein | AUC | p-value* |
|---|---|---|
| Thymosin-β4 | 0.94 | 0.066 |

*One-sided p-value versus AF volume.

TABLE 8
Cluster Ac-SDKP + AF

| Cluster | AUC | p-value* |
|---|---|---|
| Ac-SDKP + AF | 0.98 | 0.042 |

*One-sided p-value versus AF volume.

### REFERENCES

Throughout this application, various references describe the state of the art to which this invention pertains. The disclosures of these references are hereby incorporated by reference into the present disclosure.
1. Nicolaou N, Renkema K Y, Bongers E M, Giles R H, Knoers N V. Genetic, environmental, and epigenetic factors involved in CAKUT. Nature reviews Nephrology 2015; 11:720-31.
2. Calderon-Margalit R, Golan E, Twig G, et al. History of Childhood Kidney Disease and Risk of Adult End-Stage Renal Disease. N Engl J Med 2018; 378:428-38.
3. Wühl E, van Stralen K J, Verrina E, et al. Timing and outcome of renal replacement therapy in patients with congenital malformations of the kidney and urinary tract. Clin J Am Soc Nephrol 2013; 8:67-74.
4. Decramer S, Parant O, Beaufils S, et al. Anomalies of the TCF2 gene are the main cause of fetal bilateral hyperechogenic kidneys. J Am Soc Nephrol 2007; 18:923-33.
5. Morris R K, Malin G L, Khan K S, Kilby M D. Antenatal ultrasound to predict postnatal renal function in congenital lower urinary tract obstruction: systematic review of test accuracy. BJOG 2009; 116:1290-9.
6. Mehler K, Gottschalk I, Burgmaier K, et al. Prenatal parental decision-making and postnatal outcome in renal oligohydramnios. Pediatr Nephrol 2017.
7. Berry S M, Lecolier B, Smith R S, et al. Predictive value of fetal serum beta 2-microglobulin for neonatal renal function. Lancet 1995; 345:1277-8.
8. Spaggiari E, Faure G, Dreux S, et al. Sequential fetal serum beta2-microglobulin to predict postnatal renal function in bilateral or low urinary tract obstruction. Ultrasound in obstetrics & gynecology: the official journal of the International Society of Ultrasound in Obstetrics and Gynecology 2017; 49:617-22.
9. Aulbert W, Kemper M J. Severe antenatally diagnosed renal disorders: background, prognosis and practical approach. Pediatr Nephrol 2016; 31:563-74.
10. Hogan J, Dourthe M E, Blondiaux E, Jouannic J M, Garel C, Ulinski T. Renal outcome in children with antenatal diagnosis of severe CAKUT. Pediatr Nephrol 2012; 27:497-502.
11. Klein J, Lacroix C, Caubet C, et al. Fetal Urinary Peptides to Predict Postnatal Outcome of Renal Disease in Fetuses with Posterior Urethral Valves (PUV). Science translational medicine 2013; 5.
12. Desveaux C, Klein J, Leruez-Ville M, et al. Identification of Symptomatic Fetuses Infected with Cytomegalovirus Using Amniotic Fluid Peptide Biomarkers. PLoS Pathog 2016; 12:e1005395.
13. Schwartz G J, Munoz A, Schneider M F, et al. New equations to estimate GFR in children with CKD. J Am Soc Nephrol 2009; 20:629-37.
14. Benjamini Y, Hochberg Y. Controlling the false discovery rate: a practical and powerful approach to multiple testing. Journal of the Royal Statistical Society (Series B: Methodological) 1995; 57:289-300.
15. Liaw A, Wiener M. Classification and Regression by Random Forest. R News 2002; 2:18-22.
16. Mischak H, Allmaier G, Apweiler R, et al. Recommendations for biomarker identification and qualification in clinical proteomics. Science translational medicine 2010; 2:46ps2.
17. Moons K G, Kengne A P, Grobbee D E, et al. Risk prediction models: II. External validation, model updating, and impact assessment. Heart 2012; 98:691-8.
18. Loos S, Kemper M J. Causes of renal oligohydramnios: impact on prenatal counseling and postnatal outcome. Pediatr Nephrol 2018; 33:541-5.
19. Linder E, Burguet A, Nobili F, Vieux R. Neonatal renal replacement therapy: more than a technical challenge, a multidisciplinary and ethical reflection for a crucial decision. Arch Pediatr 2018; In press.
20. Payot A, Gendron S, Lefebvre F, Doucet H. Deciding to resuscitate extremely premature babies: how do parents and neonatologists engage in the decision? Soc Sci Med 2007; 64:1487-500.
21. Stevenson D K, Goldworth A. Ethical dilemmas in the delivery room. Semin Perinatol 1998; 22:198-206.
22. Lantos J D, Warady B A. The evolving ethics of infant dialysis. Pediatr Nephrol 2013; 28:1943-7.
23. Jouannic J M, Hyett J A, Pandya P P, Gulbis B, Rodeck C H, Jauniaux E. Perinatal outcome in fetuses with megacystis in the first half of pregnancy. Prenat Diagn 2003; 23:340-4.
24. Grijseels E W, van-Hornstra P T, Govaerts L C, et al. Outcome of pregnancies complicated by oligohydramnios or anhydramnios of renal origin. Prenat Diagn 2011; 31:1039-45.
25. Ryckewaert-D'Halluin A, Le Bouar G, Odent S, et al. Diagnosis of fetal urinary tract malformations: prenatal management and postnatal outcome. Prenat Diagn 2011; 31:1013-20.
26. Danziger P, Berman D R, Luckritz K, Arbour K, Laventhal N. Severe congenital anomalies of the kidney and urinary tract: epidemiology can inform ethical decision-making. Journal of perinatology: official journal of the California Perinatal Association 2016; 36:954-9.
27. Magalhaes P, Pejchinovski M, Markoska K, et al. Association of kidney fibrosis with urinary peptides: a path towards non-invasive liquid biopsies? Sci Rep 2017; 7:16915.
28. Schanstra J P, Zurbig P, Alkhalaf A, et al. Diagnosis and Prediction of CKD Progression by Assessment of Urinary Peptides. J Am Soc Nephrol 2015; 26:1999-2010.
29. Mischak H, Vlahou A, Ioannidis J P. Technical aspects and inter-laboratory variability in native peptide profiling: the CE-MS experience. Clinical biochemistry 2013; 46:432-43.
30. Morris R K, Malin G L, Quinlan-Jones E, et al. Percutaneous vesicoamniotic shunting versus conservative management for fetal lower urinary tract obstruction (PLUTO): a randomised trial. Lancet 2013; 382:1496-506.

1. A method for predicting postnatal renal function in a fetus diagnosed with bilateral congenital anomalies of the kidney and the urinary tract comprising quantifying in an amniotic fluid sample obtained from the mother the level of at least one peptide of Table A.

2. The method of claim 1 wherein the level of at least 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97 or 98 peptides from Table A is determined in the amniotic fluid sample.

3. The method of claim 1 wherein the level of peptide 31862 is determined in the amniotic fluid sample.

4. The method of claim 1 wherein the levels of 2 peptides selected from the group consisting of peptides 4697, 5420, 6196, 6400, 6600, 7437, 8721, 15510, 17010, 17207, 17264, 19221, 20228, 21320, 21342, 21353, 21684, 21830, 22456, 23894, 24856, 24868, 26070, 27115, 29894, 31787, 32876, 33930, 34055, 35853, 36447, 36627, 41269, 42122, and 45055 are determined in the amniotic fluid sample.

5. The method of claim 4 wherein the 2 peptides are selected from Table 2.

6. The method of claim 1 wherein the levels of 3 peptides selected from the group consisting of peptides 2029, 4727, 5019, 5116, 5781, 7823, 10250, 10640, 11078, 14475, 15732, 16805, 17301, 17453, 18627, 18649, 18837, 20863, 20876, 21028, 21956, 22377, 22992, 23789, 24148, 24608, 25060, 25800, 29880, 31488, 32038, 33880, 34805, 35226, 35677, 36283, 37285, 37566, 40022, and 64283 are determined in the amniotic fluid sample.

7. The method of claim 6 wherein the 3 peptides are selected from Table 3.

8. The method of claim 1 which further comprises measuring at least one clinical parameter selected from the group consisting of Age, gestational age at AF sampling; AF, amniotic fluid volume; bCAKUTPep-Age, combination of the bCAKUTPep classifier with gestational age at sampling; bCAKUTPep-AF, combination of the bCAKUTPep classifier with AF volume; bCAKUTPep-AF/Age, combination of the bCAKUTPep classifier with both gestational age at sampling and AF volume.

9. The method of claim 8 wherein the levels of 2 peptides selected from the group consisting of peptides 4727, 6400, 6600, 10786, 17760, 21342, 21684, 31862, 45055 are combined with amniotic fluid volume (AF) for predicting postnatal renal function.

10. The method of claim 9 wherein the levels of 2 peptides selected from Table 5 and amniotic fluid volume (AF) are measured for predicting postnatal renal function.

11. The method of claim 8 wherein the levels of 3 peptides selected from the group consisting of peptides 2029, 3917, 4697, 4793, 5019, 5116, 5420, 5781, 6196, 7437, 7823, 8721, 10250, 10640, 11078, 13891, 14475, 14735, 15510, 15732, 15884, 16197, 16805, 17010, 17207, 17264, 17301, 17453, 18627, 18649, 18837, 19221, 19732, 19950, 20228, 20643, 20863, 20876, 21028, 21076, 21320, 21353, 21830, 21938, 21956, 22377, 22456, 22992, 23577, 23789, 23894, 24148, 24421, 24608, 24856, 24868, 25060, 25170, 25301, 25800, 26070, 27115, 28628, 29880, 29894, 31488, 31787, 32038, 32876, 33930, 34055, 34805, 35226, 35677, 35853, 36283, 36447, 36627, 37285, 37566, 37690, 40022, 41269, 42122, 42214, 64283 are combined with amniotic fluid volume (AF) for predicting postnatal renal function.

12. The method of claim 12 wherein the levels of 3 peptides selected from Table 6 and amniotic fluid volume (AF) are measured for predicting postnatal renal function.

13. A method for predicting postnatal renal function in a fetus diagnosed with bilateral congenital anomalies of the kidney and the urinary tract comprising quantifying in an amniotic fluid sample obtained from the mother the level of thymosin-β4 or a fragment thereof.

14. The method of claim 13 wherein the level of Ac-SDKP is determined in the amniotic fluid sample.

15. The method of claim 13 wherein the fragment is selected from the group consisting of peptides 35677, 33930 and 31862 as depicted in Table A.

16. The method according to claim 1, wherein the level of the at least one peptide is determined by using a binding partner or by mass spectrometry.

17. The method of claim 1 wherein a score which is a composite of expression levels of a plurality of different peptides is determined and compared to a reference value wherein a difference between said score and said reference value indicates whether the fetus is at risk of having postnatal renal dysfunction.

18. The method of claim 1 which comprises the use of a classification algorithm selected from Linear Discriminant Analysis (LDA), Topological Data Analysis (TDA), Neural Networks, Support Vector Machine (SVM) algorithm and Random Forests algorithm (RF).

19. The method of claim 1 which comprises a) quantifying the level of a plurality of peptides of Table A in the amniotic sample; b) implementing a classification algorithm on data comprising the quantified plurality of peptides so as to obtain an algorithm output; c) determining the probability that the fetus will develop a postnatal renal dysfunction from the algorithm output of step b).

20. The method of claim 19 wherein the classification algorithm implements at least one clinical parameter selected from the group consisting of Age, gestational age at AF sampling; AF, amniotic fluid volume; bCAKUTPep-Age, combination of the bCAKUTPep classifier with gestational age at sampling; bCAKUTPep-AF, combination of the bCAKUTPep classifier with AF volume; bCAKUTPep-AF/Age, combination of the bCAKUTPep classifier with both gestational age at sampling and AF volume.

21. The method of claim 20 wherein the classification algorithm implements the amniotic fluid volume (AF).
